(19) United States
(12) Patent Application Publication
(10) Pub. No.: US 2026/0133327 A1
(43) Pub. Date: May 14, 2026

(54) RELATIVE OPTIMIZED LINEARIZATION FOR RADIOCHROMIC FILM DOSIMETRY WITH NONUNIFORMITY CORRECTION

(71) Applicant: Aktina Corp., Congers, NY (US)

(72) Inventor: Nicholas G. Zacharopoulos, West Nyack, NY (US)

(73) Assignee: Aktina Corp., Congers, NY (US)

(21) Appl. No.: 19/390,005

(22) Filed: Nov. 14, 2025

Related U.S. Application Data
(60) Provisional application No. 63/720,532, filed on Nov. 14, 2024.

(51) Int. Cl. G01T 1/06; A61N 5/10; G01T 1/29; H05H 7/00

(52) U.S. Cl. CPC G01T 1/06; A61N 5/1031; A61N 5/1065; A61N 5/1071; A61N 5/1075; G01T 1/2921; H05H 7/001; A61N 2005/1076; H05H 2007/002; H05H 2007/007; H05H 2007/008; H05H 2240/00; H05H 2277/11

(57) ABSTRACT

Systems and methods for relative optimized linearization (ROL) for radiochromic film (RCF) dosimetry may eliminate the need for dose-response curve measurements while incorporating non-uniformity corrections. The ROL method may include, for each of multiple color channels, determining linearization parameters that minimize a cost function that evaluates an error between expected and calculated doses. The ROL method may use the linearization parameters to generate a variation map including dose-independent variation values and use the variation map to determine a corrected net optical density of the RCF. The ROL method may use the linearization parameters and the corrected net optical density to produce a dose image including corrected doses.

### CROSS-REFERENCE TO RELATED APPLICATION

The present application claims the benefit of priority to U.S. Provisional Patent Application Ser. No. 63/720,532, filed on Nov. 14, 2024, which is incorporated herein by reference in its entirety.

### BACKGROUND

### Field of Invention

The present invention relates generally to radiation therapy in which a medical linear accelerator (LINAC) delivers a radiation beam to a precise point within a patient. In particular, the present invention relates to nonuniformity correction for relative film dosimetry.

### Discussion of the Background

### 1.1. Overview

Radiochromic film (RCF) dosimetry has become a widely adopted tool in radiation therapy due to its weak energy dependence, near-tissue equivalent, and high spatial resolution and ease of use. RCF dosimetry is commonly applied to verify dose distributions in patient-specific treatment plans and to conduct quality assurance (QA) on the treatment planning systems that guide medical linear accelerators (LINACs). Radiochromic films respond to radiation by darkening in response to radiation, with the degree of darkening dependent on (e.g., proportional to) the radiation dose absorbed by the RCF. Thus, RCFs enable for precise mapping of radiation dose distributions. However, achieving accurate RCF dosimetry requires accounting for various factors that can affect film response, such as thickness variations, scanner variations, and environmental conditions.

Standard approaches for RCF dosimetry often rely on absolute dosimetry techniques, which demand careful calibration for each batch of film to adjust for these dose-independent variables. Micke, D. F. Lewis, and X. Yu, Multichannel film dosimetry with nonuniformity correction, Medical Physics 38, 2523-2534 (2011) (“Micke”) outlines a method for absolute dosimetry that corrects for local disturbances, like thickness inconsistencies, that are unrelated to the delivered dose. Although effective, this approach involves calibration curves that are time-consuming to produce, adding complexity to routine clinical workflows.

To streamline this process, S. Devic, N. Tomic, S. Aldelaijan, F. DeBlois, J. Seuntjens, M. F. Chan, and D. Lewis, Linearization of dose-response curve of the radiochromic film dosimetry system, Medical Physics 39, 4850-4857 (2012) (“Devic”) introduced a relative dosimetry method that bypasses the need for batch-specific calibration. While this approach reduces amount of effort required to convert a film image to a dose map, it sacrifices accuracy by neglecting dose-independent disturbances, thus limiting its effectiveness in cases where precision is essential.

An ideal solution would involve an accurate relative dosimetry technique that can also correct for dose-independent factors, providing a balance between accuracy and efficiency.

### 1.2. Film Measurements

FIG. 1 shows a medical linear accelerator (LINAC) 100 setup for radiochromic film measurement, which can be used to verify patient treatment accuracy prior to actual treatment. In FIG. 1, a film phantom 104 is positioned on the patient couch 102 beneath the radiation beam 103. FIG. 2 shows a typical measurement setup of the film phantom 104. As shown in FIG. 2, the film phantom 104 includes a radiochromic film (RCF) 205 sandwiched between two pieces of plastic material 206. The pieces of plastic material 206 material are shown as separate from one another in FIG. 2 for clarity but, in operation, would be in contact with opposite sides of the RCF 205. After the LINAC 100 delivers treatment to the film phantom 104, the RCF 205 of the film phantom 104 is scanned by a scanner, and the resulting image is converted to a dose distribution with the methods described below. This dose distribution can then be compared with an expected dose distribution (e.g., an expected dose distribution computed by treatment planning software).

An example of an RCF 205 exposed to a square radiation field is shown in FIG. 3. In FIG. 3, the dark square at the center of the image represents the area of the RCF 205 exposed to radiation, which darkened the RCF 205 in proportion to the dose delivered. The image of FIG. 3 was acquired using a transparency scanner, which may, for example, be set to color mode with 16 bits per channel. Plotting the pixel values for the red, green, and blue color channels along the center row of the radiation field reveals the red, green, and blue profiles shown in FIG. 4. An area of ongoing research relates to the process of accurately converting these pixel values from the scanned film image into units of radiation dose.

### 1.3. Absolute Dosimetry

Absolute dosimetry requires careful batch-specific calibration to convert pixel values to dose and account for dose-independent variables. Micke introduced a multichannel dosimetry (MCD) method that utilizes all three color channels to compute dose while correcting for local variations, such as thickness inconsistencies, unrelated to the delivered dose. Micke proposed that the dose for each channel can be expressed as:

$${D}_{c , i} = {ℱ}_{c}^{- 1} ( {scanOD}_{c , i} \cdot {V}_{i} ) \qquad ( 1 )$$

where D is the computed dose, F_{c}⁻¹ is the inverse of the dose-response function, scanOD is the scanned optical density defined in Equation 2 below, V is the local dose-independent variation (e.g., non-uniform film thickness, scanner response), and the subscripts c and i indicate the color channel and the pixel location in the image, respectively.

Micke used the following definition for scanned optical density, where pixel values are normalized to the maximum value of the scanner's 16 bit output:

$${scanOD}_{c , i} = log \frac{65535}{{PV}_{c , i}} \qquad ( 2 )$$

where PV_{c,i} is the pixel value at location i and color channel c. The dose-response curve is generated by delivering known doses to films and measuring the scanner's optical density as a function of dose over a sufficiently large region of the film to average out dose-independent variations. The calibration curves generated as the dose-response function for the RGB channels for the data presented in Micke are shown in FIG. 5. Once the dose-response curve fitting parameters are determined, the variation signal can be determined through optimization, assuming dose independence across channels. The cost function for this optimization is expressed as:

$${\sum }_{j \neq k} {( {D}_{i , j} - {D}_{i , k} )}^{2} \to {min}_{{V}_{i}} . \qquad ( 3 )$$

where Dᵢ represents the dose at a pixel location i, Vᵢ denotes the variation at that same location, and j and k index the three color channels: red, green, and blue.

After determining the variation value for each pixel in the scanned film image, the variation can be removed to calculate the dose using Equation 1, with the optical density adjusted by dividing out the variation value. Shortly after the MCD technique was introduced, Lewis D, Micke A, Yu X, Chan MF. An efficient protocol for radiochromic film dosimetry combining calibration and measurement in a single scan. Med Phys. 2012; 39:6339-6350 (“Lewis”) refined the MCD technique by incorporating calibration film patches with measurement films in a single scan, reducing environmental and interscan variability. In the following, the combined one-scan MCD technique is referred to simply as MCD.

### 1.4 Relative Dose Linearization

While effective, the MCD approach requires time-consuming calibration curves and additional patches for the one-scan method, adding complexity to clinical workflows. To simplify this process, Devic introduced a relative dosimetry method that eliminates the need for both traditional calibration curves and one-scan patches by employing the following scalable pixel-to-dose linearization function:

$${D}_{c , i} = a \cdot \frac{{netOD}_{c , i}^{p}}{ln ( {netOD}_{c , i} )} \qquad ( 4 )$$

where D is the computed dose, netOD is the measured film net optical density, p is a fixed value of ⅔, and a is a scaling or normalization factor common to relative dosimetry techniques such as film, detector arrays, or ion-chamber scans such as PDDs or profiles. Devic used the net optical density representation, where each pixel is normalized not by the maximum scanner output value, but by the pixel value of an unexposed film:

$${netOD}_{c , i} = log \frac{{PV}_{c , unexp}}{{PV}_{c , i}} \qquad ( 5 )$$

where the indices i and c represent the pixel location in the image and the color channel, respectively, and PV_{c,unexp} is the averaged pixel value over a region of interest from an unexposed piece of film from the same batch as the exposed film.

Once linearized, the result is scaled based on the expected dose, as is typical in relative dosimetry. While this approach simplifies converting a film image into a dose map, it sacrifices accuracy by ignoring dose-independent variations, which can result in errors as large as 6%, thus limiting its utility for situations where accuracy is critical. Additionally, it was only demonstrated to be accurate at a specific dose range of 4 Gy, with no data confirming its applicability across lower or higher doses.

To illustrate this inaccuracy of the approach of Devic, consider the hypothetical 1D dose profile shown in FIG. 6. Using the calibration curves presented by Micke, which are shown in FIG. 5, it is possible to convert the hypothetical dose to the pixel values shown in FIG. 7. These three-channel pixel values can then be used as input for the Devic linearization technique to assess if the approach of Devic accurately reproduces the original dose profile.

Devic's linearization function was applied to all three pixel profiles, and the profiles were then normalized to the maximum dose value to produce the results shown in FIG. 8. In FIG. 8, the doses from each RGB color channel are shown along with the locally computed percent error relative to the expected dose. These results show errors exceeding the 2% threshold recommended by Devic for acceptable agreement between measured and predicted doses.

Other dose ranges were also evaluated, and preliminary results indicate that the best match occurs only within a narrow range of doses between 4-6 Gy, where the local errors stay below 2%. Accordingly, improvements are needed to more accurately convert pixel values from the scanned film image into units of radiation dose.

As noted above, an ideal solution would involve a sufficiently robust relative dosimetry technique that can also correct for dose-independent factors, providing a balance between accuracy and efficiency.

### SUMMARY

Aspects of the present invention relate to enhancing the relative dose linearization approach of Devic by varying the power function in Equation 4. Some aspects may include optimizing the numerator for each channel through a numerical search with a cost function that minimizes the difference between expected and observed doses. Using the optimized numerators achieves more accurate results than using unoptimized numerators.

Some aspects may compute variation maps for non-uniformity correction without dose-response curves. In some aspects, this may be accomplished by first improving the accuracy of existing linearization techniques and then by replacing the dose-response function (F⁻¹ in Equation 1) with the improved linearization. This may allow for the cost function in Equation 3 to be cast solely via relative film dosimetry techniques.

One aspect of the invention may provide a method including, for each color channel c of multiple color channels, for each pixel location i of a digital image of a radiochromic film (RCF) that was exposed to radiation generated by a linear accelerator (LINAC), using a pixel value PV_{c,i} at the pixel location i to calculate a dose D_{c,i} of radiation in the color channel c absorbed by the RCF at a location corresponding to the pixel location i. An optical density of the RCF at the location may have changed in response to the dose of the radiation absorbed by the RCF at the location. The method may include, for each color channel c of the multiple color channels, determining linearization parameters that minimize a cost function that evaluates an error between expected doses Dᵢ at pixel locations i and the calculated doses D_{c,i} of the radiation in the color channel c absorbed by the RCF at the locations corresponding to the pixel locations i. The method may include generating a variation map including, for each pixel location i of the digital image of the RCF, a dose-independent variation value Vᵢ for the pixel location i. Generating the variation map may include, for each pixel location i of the digital image of the RCF: (i) for each color channel c of the multiple color channels, using the linearization parameters determined for the color channel c to calculate an optimized linearized dose D^{rol}_{c,i} of the radiation in the color channel c absorbed by the RCF at the location corresponding to the pixel location i, and (ii) determining the variation value Vᵢ for the pixel location i based on differences between the optimized linearized doses D^{rol}_{c,i} calculated for the multiple color channels c. The method may include, for each color channel c of the multiple color channels, for each pixel location i of the digital image of the RCF, using the pixel value PV_{c,i} for the pixel location i in the color channel c and the variation value Vi for the pixel location i to determine a corrected net optical density netOD^{corrected}_{c,i} of the RCF for the color channel c at the pixel location i. The method may include producing a dose image including a corrected dose for each color channel c of the multiple color channels at each pixel location i of the digital image of the RCF. Producing the dose image may include, for each color channel c of the multiple color channels and each pixel location i of the digital image of the RCF, using the linearization parameters determined for the color channel c and the corrected net optical density netOD^{corrected}_{c,i} of the RCF for the color channel c at the pixel location i to calculate the corrected dose for the color channel c at the pixel location i.

In some aspects, the multiple color channels may include red, green, and blue channels.

In some aspects, the method may further include using the LINAC to generate the radiation and expose the RCF to the radiation.

In some aspects, the method may further include using a scanner to scan the RCF across the multiple color channels to generate the digital image of the RCF with responses of the RCF to the dose of absorbed radiation in the multiple color channels.

In some aspects, for each color channel c of the multiple color channels, for each pixel location i of the digital image of the RCF that was exposed to the radiation, using the pixel value PV_{c,i} at the pixel location i to calculate the dose D_{c,i} of radiation in the color channel c absorbed by the RCF at the location corresponding to the pixel location i may include: (i) using the pixel value PV_{c,i} at the pixel location i to determine an uncorrected net optical density netOD_{c,i} of the RCF in the color channel c at the location of the RCF corresponding to the pixel location i and (ii) using the uncorrected net optical density netODc,i of the RCF in the color channel c at the location of the RCF corresponding to the pixel location i to calculate the dose D_{c,i} of radiation in the color channel c absorbed by the RCF at the location corresponding to the pixel location i. In some aspects, using the pixel value PV_{c,i} at the pixel location i to determine the uncorrected net optical density netOD_{c,i} of the RCF in the color channel c at the location of the RCF corresponding to the pixel location i may include normalizing the pixel value PV_{c,i} at the pixel location i by an averaged pixel value PV_{c,unexp} of a digital image of an unexposed RCF in the color channel c. In some aspects, the method may further include using a scanner to scan the unexposed RCF across the multiple color channels to generate the digital image of the unexposed RCF.

In some aspects, the linearization parameters for each color channel c may include a scaling value a_{c} and a power value p_{c}.

In some aspects, the error evaluated by the cost function may be a mean absolute error between the expected doses Dᵢ at the pixel locations i and the calculated doses D_{c,i} of the radiation in the color channel c absorbed by the RCF at the locations corresponding to the pixel locations i.

In some aspects, the corrected net optical density netOD^{corrected}_{c,i} of the RCF in the color channel c at the pixel location i may be determined using an averaged pixel value PV_{c,unexp} of a digital image of an unexposed RCF in the color channel c. In some aspects, determining the corrected net optical density netOD^{corrected}_{c,i} of the RCF for the color channel c at the pixel location i using the averaged pixel value PV_{c,unexp} of the digital image of the unexposed RCF in the color channel c may include: (i) using the averaged pixel value PV_{c,unexp} of the digital image of the unexposed RCF in the color channel c to calculate a scanned optical density scanOD_{c,unexp} of the digital image of the unexposed RCF in the color channel c and (ii) using the scanned optical density scanOD_{c,unexp} of the digital image of the unexposed RCF in the color channel c to determine the corrected net optical density netOD^{corrected}_{c,i} of the RCF in the color channel c at the pixel location i.

In some aspects, for each color channel c of the multiple color channels, for each pixel location i of the digital image of the RCF, using the pixel value PV_{c,i} at the pixel location i in the color channel c and the variation value Vᵢ for the pixel location i to determine a corrected net optical density netOD^{corrected}_{c,i} of the RCF in the color channel c at the location of the RCF corresponding to the pixel location i may include: (i) using the pixel value PV_{c,i} at the pixel location i in the color channel c to calculate a scanned optical density scanODc,i for the pixel location i in the color channel c and (ii) using the scanned optical density scanODc,i for the pixel location i in the color channel c and the variation value Vᵢ for the pixel location i to determine the corrected net optical density netOD^{corrected}_{c,i} of the RCF in the color channel c at the location of the RCF corresponding to the pixel location i.

In some aspects, the method may further include comparing the corrected doses with the expected doses Di and determining that the LINAC meets acceptable performance criteria. In some aspects, the method may further include exposing a patient to treatment radiation generated by the LINAC only if the LINAC was determined to meet the acceptable performance criteria.

In some aspects, the method may further include using the dose image to recalibrate the LINAC and using the recalibrated LINAC to generate treatment radiation that exposes a patient to a radiation dose distribution in accordance with an expected dose distribution. In some aspects, recalibrating the LINAC includes determining adjustments to the LINAC that would cause the LINAC to generate radiation that would expose the patient to a radiation dose distribution in accordance with the expected dose distributions and making the determined adjustments to the LINAC. In some aspects, recalibrating the LINAC includes adjusting the expected dose distribution to match the dose image.

Another aspect of the invention may provide an apparatus. The apparatus may be configured to, for each color channel c of multiple color channels, for each pixel location i of a digital image of a radiochromic film (RCF) that was exposed to radiation generated by a linear accelerator (LINAC), use a pixel value PV_{c,i} at the pixel location i to calculate a dose D_{c,i} of radiation in the color channel c absorbed by the RCF at a location corresponding to the pixel location i. An optical density of the RCF at the location may have changed in response to the dose of the radiation absorbed by the RCF at the location. The apparatus may be configured to, for each color channel c of the multiple color channels, determine linearization parameters that minimize a cost function that evaluates an error between expected doses Dᵢ at pixel locations i and the calculated doses D_{c,i} of the radiation in the color channel c absorbed by the RCF at the locations corresponding to the pixel locations i. The apparatus may be configured to generate a variation map including, for each pixel location i of the digital image of the RCF, a dose-independent variation value Vᵢ for the pixel location i. Generating the variation map may include, for each pixel location i of the digital image of the RCF: (i) for each color channel c of the multiple color channels, using the linearization parameters determined for the color channel c to calculate an optimized linearized dose D^{rol}_{c,i} of the radiation in the color channel c absorbed by the RCF at the location corresponding to the pixel location i and (ii) determining the variation value Vᵢ for the pixel location i based on differences between the optimized linearized doses D^{rol}_{c,i} calculated for the multiple color channels c. The apparatus may be configured to, for each color channel c of the multiple color channels, for each pixel location i of the digital image of the RCF, use the pixel value PV_{c,i} for the pixel location i in the color channel c and the variation value Vᵢ for the pixel location i to determine a corrected net optical density netOD^{corrected}_{c,i} of the RCF for the color channel c at the pixel location i. The apparatus may be configured to produce a dose image comprising a corrected dose for each color channel c of the multiple color channels at each pixel location i of the digital image of the RCF. Producing the dose image may include, for each color channel c of the multiple color channels and each pixel location i of the digital image of the RCF, using the linearization parameters determined for the color channel c and the corrected net optical density netOD^{corrected}_{c,i} of the RCF for the color channel c at the pixel location i to calculate the corrected dose for the color channel c at the pixel location i.

In some aspects, the multiple color channels may include red, green, and blue channels.

In some aspects, the apparatus may be further configured to use the LINAC to generate the radiation and expose the RCF to the radiation.

In some aspects, for each color channel c of the multiple color channels, for each pixel location i of the digital image of the RCF that was exposed to the radiation, using the pixel value PV_{c,i} at the pixel location i to calculate the dose D_{c,i} of radiation in the color channel c absorbed by the RCF at the location corresponding to the pixel location i may include: (i) using the pixel value PV_{c,i} at the pixel location i to determine an uncorrected net optical density netOD_{c,i} of the RCF in the color channel c at the location of the RCF corresponding to the pixel location i and (ii) using the uncorrected net optical density netOD_{c,i} of the RCF in the color channel c at the location of the RCF corresponding to the pixel location i to calculate the dose D_{c,i} of radiation in the color channel c absorbed by the RCF at the location corresponding to the pixel location i.

In some aspects, using the pixel value PV_{c,i} at the pixel location i to determine the uncorrected net optical density netOD_{c,i} of the RCF in the color channel c at the location of the RCF corresponding to the pixel location i may include normalizing the pixel value PV_{c,i} at the pixel location i by an averaged pixel value PV_{c,unexp} of a digital image of an unexposed RCF in the color channel c.

In some aspects, the linearization parameters for each color channel c may include a scaling value a_{c} and a power value p_{c}.

In some aspects, the error evaluated by the cost function may be a mean absolute error between the expected doses Dᵢ at the pixel locations i and the calculated doses D_{c,i} of the radiation in the color channel c absorbed by the RCF at the locations corresponding to the pixel locations i.

In some aspects, the corrected net optical density netOD^{corrected}_{c,i} of the RCF in the color channel c at the pixel location i may be determined using an averaged pixel value PV_{c,unexp} of a digital image of an unexposed RCF in the color channel c. In some aspects, determining the corrected net optical density netOD^{corrected}_{c,i} of the RCF for the color channel c at the pixel location i using the averaged pixel value PV_{c,unexp} of the digital image of the unexposed RCF in the color channel c may include: (i) using the averaged pixel value PV_{c,unexp} of the digital image of the unexposed RCF in the color channel c to calculate a scanned optical density scanOD_{c,unexp} of the digital image of the unexposed RCF in the color channel c and (ii) using the scanned optical density scanOD_{c,unexp} of the digital image of the unexposed RCF in the color channel c to determine the corrected net optical density netOD^{corrected}_{c,i} of the RCF in the color channel c at the pixel location i.

In some aspects, for each color channel c of the multiple color channels, for each pixel location i of the digital image of the RCF, using the pixel value PV_{c,i} at the pixel location i in the color channel c and the variation value Vᵢ for the pixel location i to determine a corrected net optical density netOD^{corrected}_{c,i} of the RCF in the color channel c at the location of the RCF corresponding to the pixel location i may include: (i) using the pixel value PV_{c,i} at the pixel location i in the color channel c to calculate a scanned optical density scanODc,i for the pixel location i in the color channel c and (ii) using the scanned optical density scanODc,i for the pixel location i in the color channel c and the variation value Vᵢ for the pixel location i to determine the corrected net optical density netOD^{corrected}_{c,i} of the RCF in the color channel c at the location of the RCF corresponding to the pixel location i.

In some aspects, the apparatus may be further configured to compare the corrected doses with the expected doses Di and determine whether the LINAC meets acceptable performance criteria, wherein a patient is exposed to treatment radiation generated by the LINAC only if the LINAC was determined to meet the acceptable performance criteria.

Yet another aspect of the invention may provide a system including the apparatus, the LINAC, and a scanner.

Still another aspect of the invention may provide a method. The method may include scanning a radiochromic film (RCF) across multiple color channels to generate a digital image. An optical density of the RCF may change as a result of radiation exposure, the RCF may contain an area exposed to a pattern of radiation doses, and the digital image may include responses of the RCF in multiple color channels. The method may include determining an optimal mathematical function that linearizes and scales a response of the RCF in each of the multiple color channels so that a transformed pixel response of each of the multiple color channels is optimally matched to an expected dose distribution for the RCF. The method may include applying the optimal mathematical function to determine a contribution of the dose-independent portion, wherein removing the dose-independent portion minimizes differences in radiation dose values across the multiple color channels. The method may include removing the dose-independent portion from the color channels to produce a dose image that reflects only dose-dependent values.

Further variations encompassed within the systems and methods are described in the detailed description of the invention below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated herein and form part of the specification, illustrate various, non-limiting embodiments of the present invention. In the drawings, like reference numbers indicate identical or functionally similar elements.

FIG. 1 illustrates a medical linear accelerator (LINAC).

FIG. 2 illustrates a film phantom including a radiochromic film (RCF).

FIG. 3 illustrates an example image of radiochromic film that has been exposed to a square field of radiation.

FIG. 4 illustrates pixel values across a profile through the center of the image shown in FIG. 3.

FIG. 5 illustrates calibration curves for data presented in Micke.

FIG. 6 illustrates a hypothetical dose profile used to demonstrate the inaccuracy of the Devic linearization technique.

FIG. 7 illustrates simulated RGB pixel values determined from the calibration and dose curves of FIGS. 5 and 6.

FIG. 8 illustrates dose linearization using Devic's formula, with locally computed percent errors shown as dashed lines on the right-side vertical axis.

FIG. 9 illustrates a system according to some aspects.

FIG. 10 illustrates the apparatus of the system according to some aspects.

FIG. 11 illustrates a process according to some aspects.

FIG. 12 illustrates a process according to some aspects.

FIGS. 13A and 13B illustrate simulation results for 3 and 10 Gy dose profiles, respectively, based on measured EBT4 dose-response curves, with all lines representing the global percentage error between the original input and linearized output doses for each channel, with dashed lines indicate the non-optimized linearization and solid lines depicting the optimized results, and with each line being color-coded to match its corresponding color channel.

FIG. 14 illustrates average linearization absolute error per channel as a function of maximum dose for EBT4 film across all three color channels for simulations ranging from 1 to 10 Gy, with dashed lines representing non-optimized linearization and solid lines indicating optimized average errors.

FIG. 15 illustrates variation maps generated using both the multichannel dosimetry (MCD) and the ROL methods for the wedge test plan.

FIG. 16 illustrates global percent error maps comparing ROL to MCD computed doses for all three color channels in: (a) open field, (b) wedge field, (c) volumetric modulated arc therapy (VMAT) plan, (d) VMAT plan with a 3 mm shift, and (e) partially delivered VMAT plan.

FIG. 17 illustrates global percent error maps comparing ROL to MCD computed doses for the red color channel for different linearization optimization threshold values (r in Equation 6) for all three test plans.

FIGS. 18A and 18B illustrate measured versus expected red channel dose profiles (cross-plane and in-plane, respectively) through the center of the upper-right target of the VMAT plan, as indicated by the crosshairs in FIG. 21A, with the MCD dose shown in orange, the ROL dose in maroon, and the expected dose from the planning system in gray.

FIGS. 19A and 19B illustrate measured versus expected red channel dose profiles (cross-plane and in-plane, respectively) through the center of the upper-right target of the partial VMAT plan, as indicated by the crosshairs in FIG. 21A, with the MCD dose is shown in orange, the ROL dose in maroon, and the expected dose from the planning system in gray.

FIG. 20 illustrates red channel gamma analysis maps (3%, 2 mm) generated using both MCD and ROL methods for the axial film measurement of the TG-119 C-Shape treatment plan (6 MV), across MLC offset values ranging from 0.0 to 0.4 mm.

FIGS. 21A-21D illustrate gamma analysis maps (3%, 2 mm) for VMAT expected dose compared against (a) fully delivered film analyzed with MCD, (b) fully delivered film analyzed with ROL, (c) shifted film analyzed with MCD, and (d) shifted film analyzed with ROL, with the crosshairs in FIG. 21A indicating the location of the profiles shown in FIGS. 18A-19B.

### DETAILED DESCRIPTION OF PREFERRED EMBODIMENTS

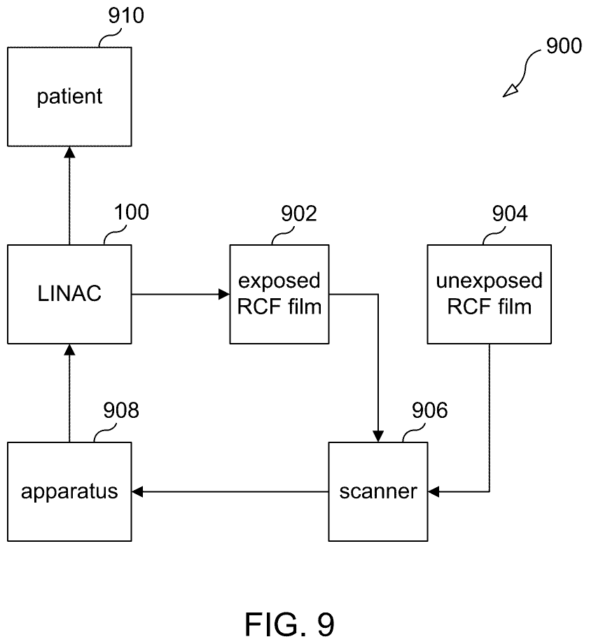

In some aspects, as shown in FIG. 9, a system 900 may include a medical linear accelerator (LINAC) 100, a scanner 906, and an apparatus 908. In some aspects, the apparatus 908 may be separate from the LINAC 100. However, this is not required, and, in some alternative aspects, the LINAC 100 may include all or a portion of the apparatus 908 (e.g., the apparatus 908 may be a LINAC controller). In some aspects, the apparatus 908 may be separate from the scanner 906. However, this is not required, and, in some alternative aspects, the scanner 906 may include all or a portion of the apparatus 908.

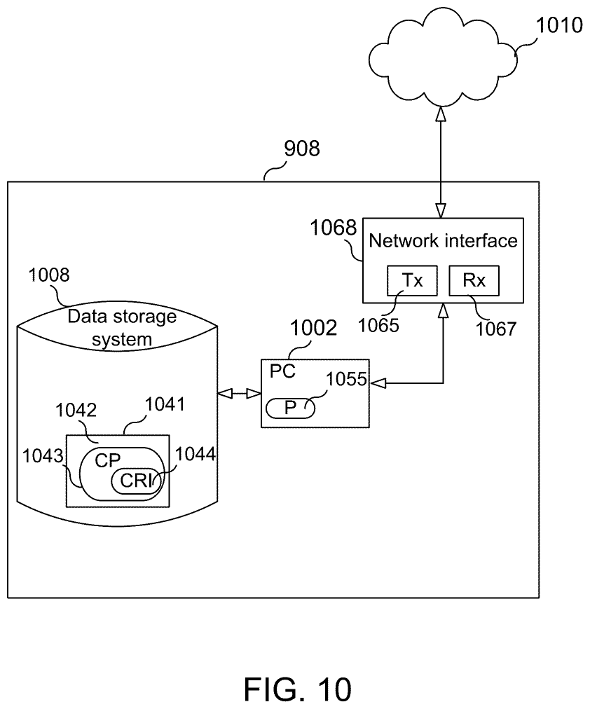

FIG. 10 is a block diagram of the apparatus 908 according to some aspects. As shown in FIG. 10, the apparatus 908 may include: processing circuitry (PC) 1002, which may include one or more processors (P) 1055 (e.g., one or more general purpose microprocessors and/or one or more other processors, such as an application specific integrated circuit (ASIC), field-programmable gate arrays (FPGAs), and the like), which processors may be co-located in a single housing or in a single data center or may be geographically distributed (i.e., the system may be a distributed computing apparatus); a network interface 1068 comprising a transmitter (Tx) 1065 and a receiver (Rx) 1067 for enabling the apparatus 908 to transmit data to and receive data from other nodes (e.g., the LINAC 100 and/or the scanner 906) connected to a network 1010 (e.g., an Internet Protocol (IP) network) to which network interface 1068 is connected; and a local storage unit (a.k.a., “data storage system”) 1008, which may include one or more non-volatile storage devices and/or one or more volatile storage devices. In some aspects in which the PC 1002 includes a programmable processor, a computer program product (CPP) 1041 may be provided. In some aspects, the CPP 1041 may include a computer readable medium (CRM) 1042 storing a computer program (CP) 1043 comprising computer readable instructions (CRI) 1044. In some aspects, the CRM 1042 may be a non-transitory computer readable medium, such as, magnetic media (e.g., a hard disk), optical media, memory devices (e.g., random access memory, flash memory), and the like. In some aspects, the CRI 1044 of computer program 1043 may be configured such that when executed by PC 1002, the CRI causes the apparatus 908 to perform steps described herein (e.g., one or more steps described herein with reference to the flowcharts herein). In other aspects, the apparatus 908 may be configured to perform steps described herein without the need for code. That is, for example, the PC 1002 may consist merely of one or more ASICs. Hence, the features of the aspects described herein may be implemented in hardware and/or software.

In some aspects, the apparatus 908 may be configured to perform relative optimized linearization (ROL). In some aspects, the ROL may improve the approach of Devic by introducing a variable power function where the values of a and p in Equation 4 can be determined numerically via optimization using a cost function that evaluates the mean absolute error between the expected and the calculated doses for each color channel:

$${\sum }_{i \vert {D}_{i} > τ} \vert {D}_{i} - {a}_{c} \cdot \frac{{netOD}_{c , i}^{p}}{ln ( {netOD}_{c , i} )} \vert \to {min}_{{a}_{c} , {p}_{c}} . \qquad ( 6 )$$

where Dᵢ is the expected dose at pixel i, obtained, for example, from a treatment planning system (TPS); netOD_{c,i} is the measured net optical density at pixel i for color channel c, and τ is a minimum dose threshold, which may prevent the optimization from disproportionately prioritizing large low-dose regions outside the radiation field. In this form of the cost function, both the power value p_{c} and the scaling value a_{c} are optimized simultaneously for each color channel c, resulting in a linearization and normalization that aims to match the measurement to the expected dose across all pixels where the expected dose is above the threshold τ. With regard to the scaling value a, this approach differs from traditional normalization methods, such as single-point maximum value or central-axis normalization, which are less robust due to potential film non-uniformity at the normalization point.

The multichannel dosimetry (MCD) method may utilize all three color channels to account for non-uniformities that introduce dose-independent film responses. The effectiveness of the MCD approach may depend on the extent to which all three channels accurately represent these non-uniformities so they can be properly corrected in the final result. This dependence may explain why relative dosimetry has not been applied to the film non-uniformity problem. For example, Devic reported that the red channel may not linearize well at doses exceeding 1 Gy. Moreover, because only the coefficients of determination (R2) for the linear fit were provided, and not the resulting linearization errors, it is difficult to fully evaluate the effectiveness of the technique from Devic.

To assess the feasibility of replacing the MCD method with a relative approach that does not require acquiring dose-response curves, the accuracy of the linearization method proposed by Devic was evaluated through simulations based on EBT4 (Ashland Inc., Wayne, NJ, USA) film dose-response data. This evaluation, which first used the original fixed power value p of ⅔, aimed to verify that the linearization process was sufficiently robust—maintaining accuracy within 1% across all three color channels, to provide reliable inputs for optimizing non-uniformity correction. The simulations were then repeated with optimized (non-fixed) power value and the results were compared to evaluate the effectiveness of the proposed technique.

The evaluation was based on a simulated one-dimensional linear dose array, Dₑₓₚ, ranging from 10 cGy to 3 Gy in 3 cGy increments. This uniformly increasing set of dose values was first converted into red, green, and blue pixel values using measured EBT4 dose-response curves, using the following rational function:

$$scanOD = log \frac{c + bD}{c + D} \qquad ( 7 )$$

where scanOD is the scanner optical density determined using Equation 2, D is the delivered dose, and a, b, and c are the fitting parameters. The fit parameters for this simulation were determined from films irradiated with a 6 MV flattened beam at 0, 25, 50, 75, 100, 150, 200, 300, 400, 500, 800, and 1000 cGy. Pixel values were obtained by averaging regions of interest (ROIs) approximately 2×2 cm in size from each film patch.

All three arrays of pixel values were then transformed into net optical density (netOD) and then linearized using the second term in Equation 4. Scaling factors a for each color channel were then applied to ensure that the maximum linearized netOD for each channel matched the maximum of the original dose profile, yielding the corrected dose profile Dₗᵢₙ. The results were compared to the original dose values to assess the technique's ability to linearize the channel-specific non-linear dose-response of EBT4 film, using global percentage errors (εᵢ) computed across all three color channels as follows:

$${ε}_{i} = \frac{{D}_{exp , i} - {D}_{lin , i}}{{D}_{exp , max}} \times 100 \qquad ( 8 )$$

where D_{exp,i} is the input dose value at location i, Dlin,i is the output linearized pixel response at location i, and D_{exp,max} is the maximum value in the input dose profile. This simulation was also repeated for a dose profile with maximum dose of 10 Gy. These dose ranges for these two initial simulations were selected to represent a typical clinical QA plan and the maximum dose specified in the EBT4 film datasheet.

While the initial simulations assessed linearization accuracy at specific maximum doses of 3 and 10 Gy, additional simulations were conducted to evaluate robustness across a broader range of maximum dose values, from 2 to 10 Gy in 10 cGy increments. Because the slope of the dose-response curve varies across the applicable dose range, and the maximum dose is commonly used as the normalization reference, this value may have an impact on the overall accuracy of the linearization process (as also reported by Devic). For each profile with a different maximum dose, a linearization error profile was computed using Equation 8, and the average error across the entire profile was calculated. This analysis provides a more comprehensive understanding of the method's performance when applied to measured films with varying maximum dose levels.

To extend the new ROL technique to correct for dose-independent non-uniformities, the optimization framework of Micke (Equation 3) was modified by replacing the dose computed using MCD (Equation 1) with the dose computed using ROL (Equation 4, using optimized values of a and p).

$${\sum }_{j \neq k} ( {D}_{i , j}^{rol} - {D}_{i , k}^{rol} ) \to {min}_{{V}_{i}} . \qquad ( 9 )$$

where D^{rol}_{i,j} is the optimized linearized dose formula modified to incorporate local dose-independent variations as follows:

$${D}_{c , i}^{rol} = {a}_{c} \cdot \frac{{netOD}_{c , i}^{p}}{ln ( {netOD}_{c , i} )} \qquad ( 10 a )$$

where a_{c} and p_{c} are the optimized scale and power values for color channel c, and netOD_{c,i} is the net optical density represented in terms of scanner optical density scanOD and the variation Vᵢ as at pixel location i and color channel c. In Equation 10a, the net optical density netOD_{c,i} is represented in terms of scanner optical density scanOD_{c,i} and the variation Vᵢ at pixel location i and color channel c as:

$${netOD}_{c , i} = {scanOD}_{c , i} \cdot {V}_{i} - {scanOD}_{c , unexp} \qquad ( 10 b )$$

As a result, the net optical density netOD_{c,i} incorporates the non-uniformity correction based on scanOD directly into the relative optimized linearized dose (based on netOD). To compute the net optical density netOD_{c,i} within this framework, as shown in Equation 10 b, each raw pixel value may be converted to scanner optical density (scanOD) using Equation 2.

After the variation value Vᵢ is calculated using Equation 9 (and Equations 10a and 10b), as shown in Equation 11, the scanner optical density scanOD_{c,i} may then be corrected for non-uniformities on a pixel-by-pixel basis by multiplying it by the variation value Vᵢ. As is also shown in Equation 11, the corrected scanner optical density (i.e., scanOD_{c,i}·Vᵢ) may then converted back to a pixel value using the inverse of Equation 2, and finally converted to a corrected net optical density (netOD) using Equation 5. This corrected netOD (shown in Equation 11) may account for the dose-independent variations consistently with the MCD method while being incorporated into the new ROL approach.

$${netOD}_{c , i}^{corrected} = {scanOD}_{c , i} \cdot {V}_{i} - {scanOD}_{c , unexp} \qquad ( 11 )$$

Equation 11 may be derived as follows:

$$\begin{matrix}{netOD}_{c , i}^{corrected} = log ( \frac{{PV}_{c , unexp}}{{PV}_{c , i}^{corrected}} ) \\ = log \frac{{PV}_{c , unexp} \cdot {10}^{{scanOD}_{c , i} \cdot {V}_{i}}}{65535} \\ = log ( {PV}_{c , unexp} ) + {scanOD}_{c , i} \cdot {V}_{i} - log ( 65535 ) \\ = {scanOD}_{c , i} \cdot {V}_{i} + log \frac{{PV}_{c , unexp}}{65535} \\ = {scanOD}_{c , i} \cdot {V}_{i} - {scanOD}_{c , unexp}\end{matrix}$$

The corrected netOD and the optimized scale and power values a_{c} and p_{c} may then be used to calculate corrected doses as shown in Equation 12:

$${D}_{c , i}^{corr} = {a}_{c} \cdot \frac{{netOD}_{c , i}^{{corr}^{{p}_{c}}}}{ln ( {netOD}_{c , i} )} , \qquad ( 12 )$$

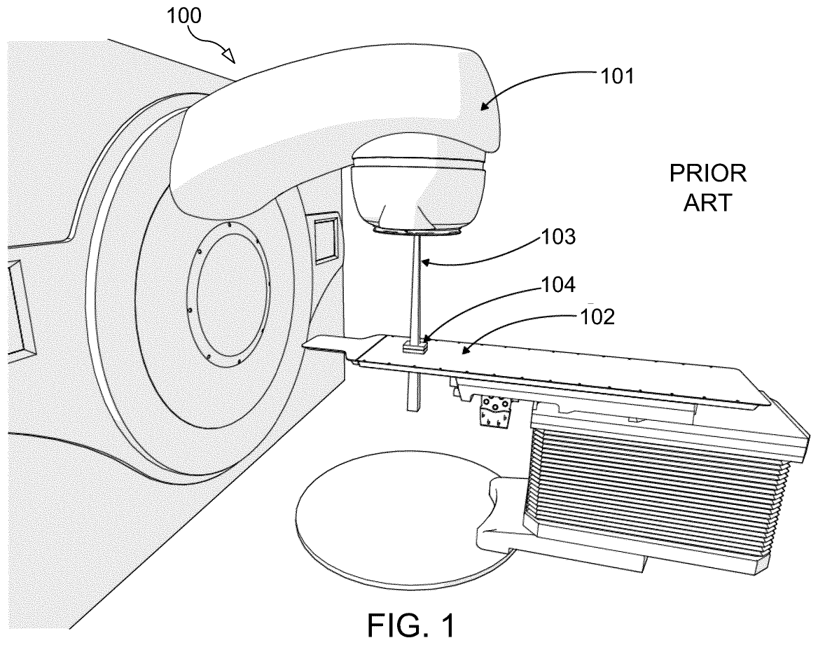

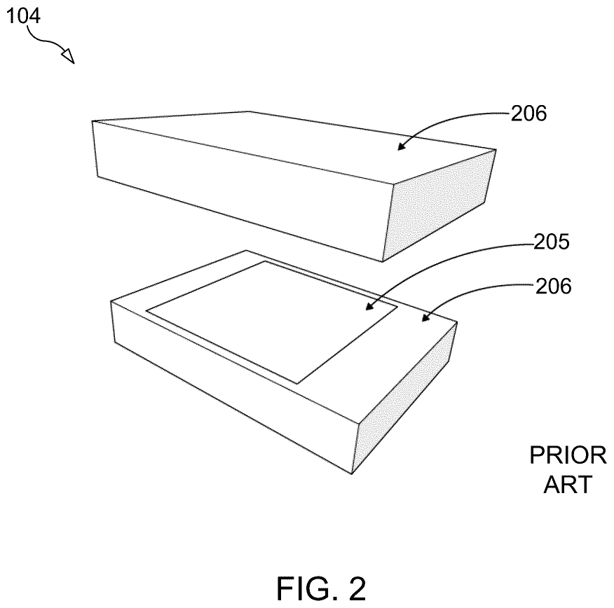

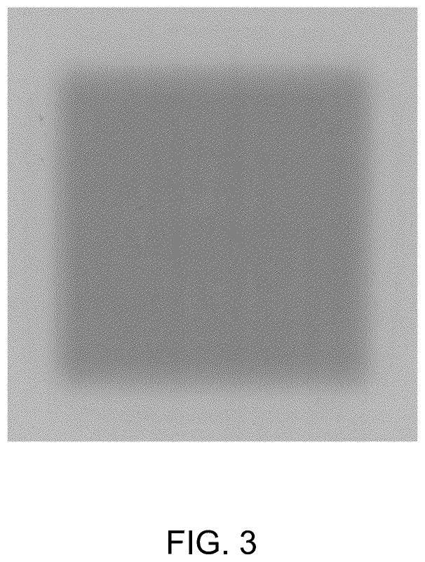

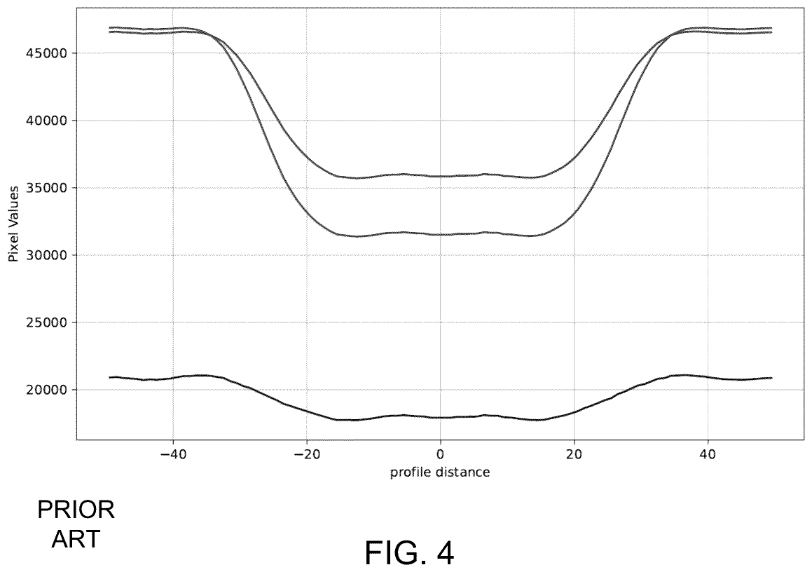

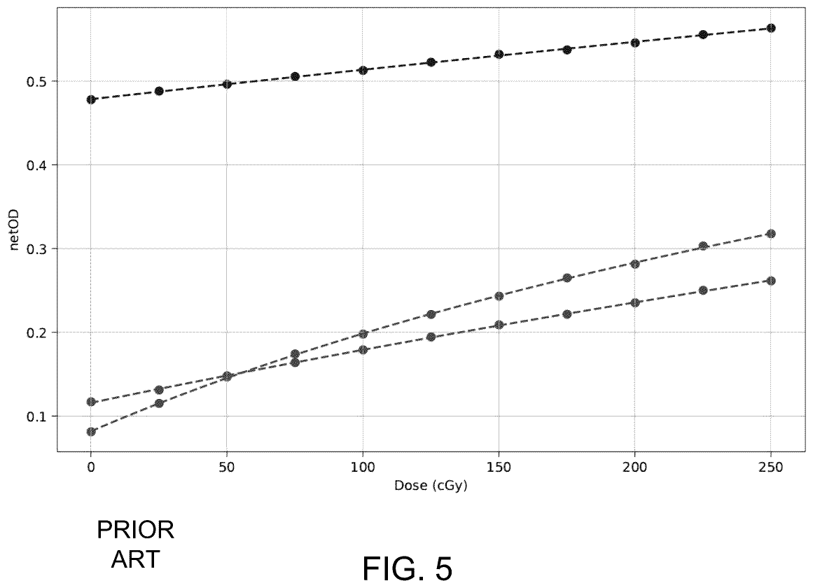

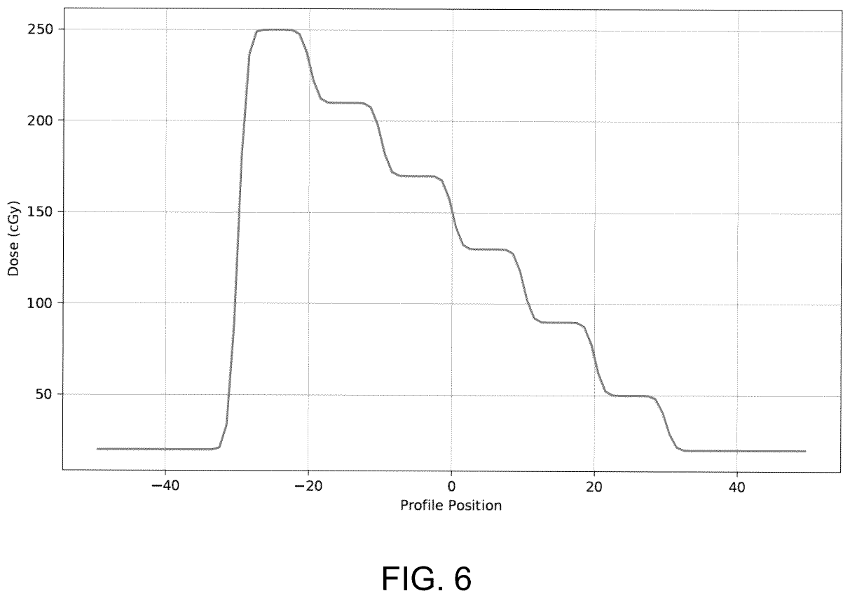

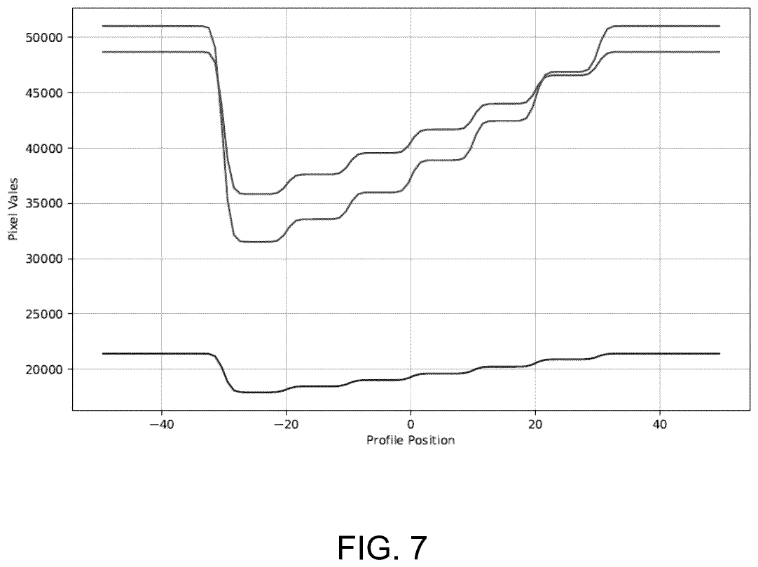

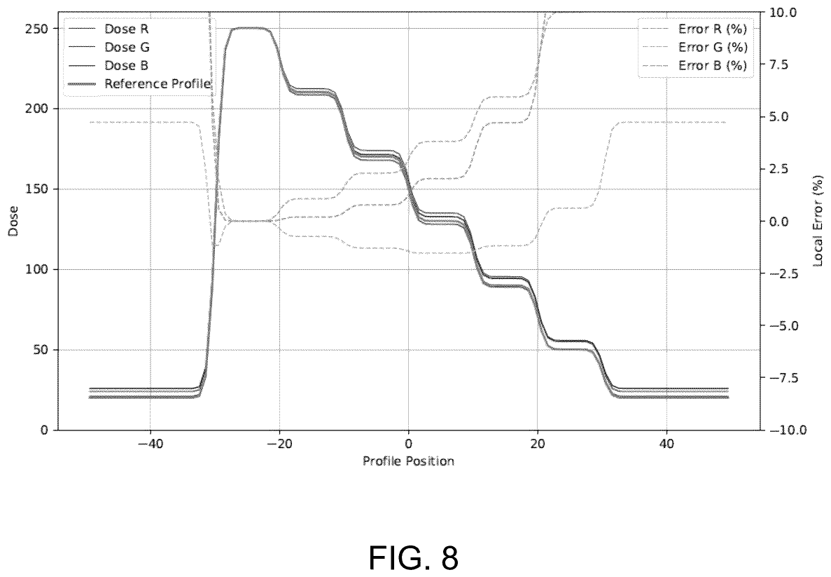

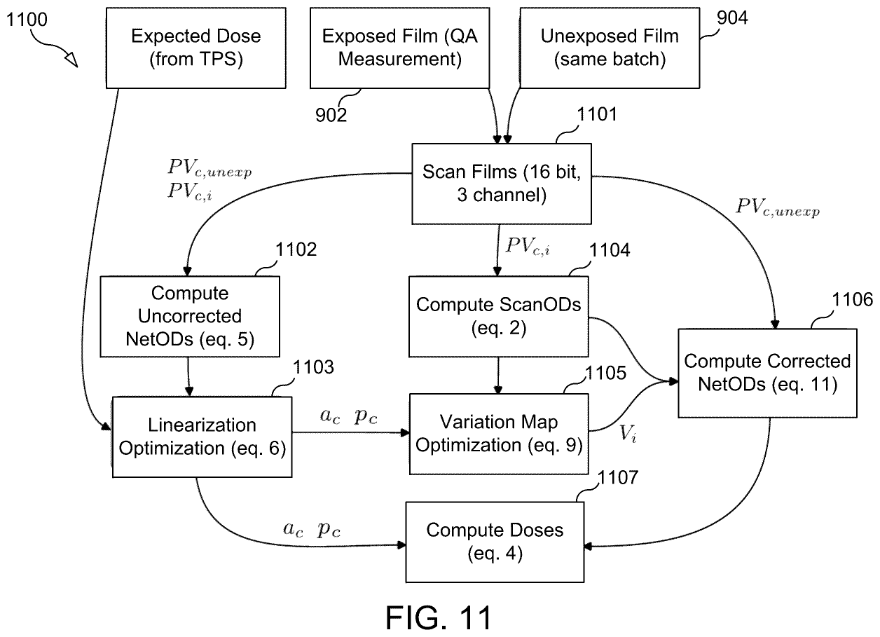

FIG. 11 is a flowchart illustrating an ROL process 1100 according to some aspects. In some aspects, one or more of the steps of the process 1100 may be performed by the system 900. In some aspects, the process 1100 may include an initial step of using the LINAC 100 to generate radiation (e.g., radiation beam 103) and expose a radiochromic film (RCF) 205 (e.g., of a film phantom 104) to the radiation. In some aspects, the LINAC 100 and film phantom 104 may be setup as shown in FIG. 1 during the initial step of the process 1100. In some aspects, the initial step may produce an exposed RCF 902.

In some aspects, as shown in FIG. 11, the process 1100 may include a step 1101 of using a scanner 906 to scan the exposed RCF 902 across multiple color channels to generate a digital image of the exposed RCF 902 with responses of the RCF 902 to a dose of absorbed radiation in the multiple color channels. In some aspects, the multiple color channels may include red, green, and blue channels. In some aspects, the step 1101 may including using the scanner 906 to scan an unexposed RCF 904 across the multiple color channels to generate a digital image of the unexposed RCF 904. In some aspects, the scanner 906 may scan the exposed RCF 902 and the unexposed RCF 904 together.

In some aspects, as shown in FIG. 11, the process 1100 may include a step 1102 of computing netOD for each color channel c using Equation 5. In some aspects, the apparatus 908 may compute the netOD for each color channel c.

In some aspects, as shown in FIG. 11, the process 1100 may include a step 1103 of determining the linearization parameters a and p for each channel c by minimizing the cost function in Equation 6. In some aspects, the apparatus 908 may determine the linearization parameters a and p for each channel c.

In some aspects, as shown in FIG. 11, the process 1100 may include a step 1104 of computing scanOD for each channel c using Equation 2. In some aspects, the apparatus 908 may compute the scanOD for each channel c.

In some aspects, the process 1100 may include a step 1105 of computing a variation map using the scanOD and the linearization parameters determined in steps 1102 and 1103, respectively, via optimization with Equation 3. In some aspects, Equation 10 b may be applied within the optimization to convert between scanOD and netOD, because the linearization may be defined in terms of netOD while the non-uniformities may be corrected using scanOD. In some aspects, the variation map may include a variation Vᵢ at each pixel location i. In some aspects, the variation map may be calculated using Equations 9, 10a, and 10b. In some aspects, the apparatus 908 may compute the variation map.

In some aspects, the process 1100 may include a step 1106 of computing a corrected netOD using Equation 11 together with scanODs and the previously determined variation map. In some aspects, the apparatus 908 may compute the corrected netOD.

In some aspects, the process 1100 may include a step 1107 of computing the corrected dose for each channel c using Equation 12, the corrected netOD from step 1106, and the linearization parameters from step 1103. In some aspects, the apparatus 908 may compute the corrected dose for each channel c.

In some aspects, the process 1100 may include a step of applying the computed corrected doses. In some aspects, the application step may include using the corrected doses to control the LINAC 100 to generate treatment radiation that exposes a patient 910 to a radiation dose distribution in accordance with a patient-specific treatment plan.

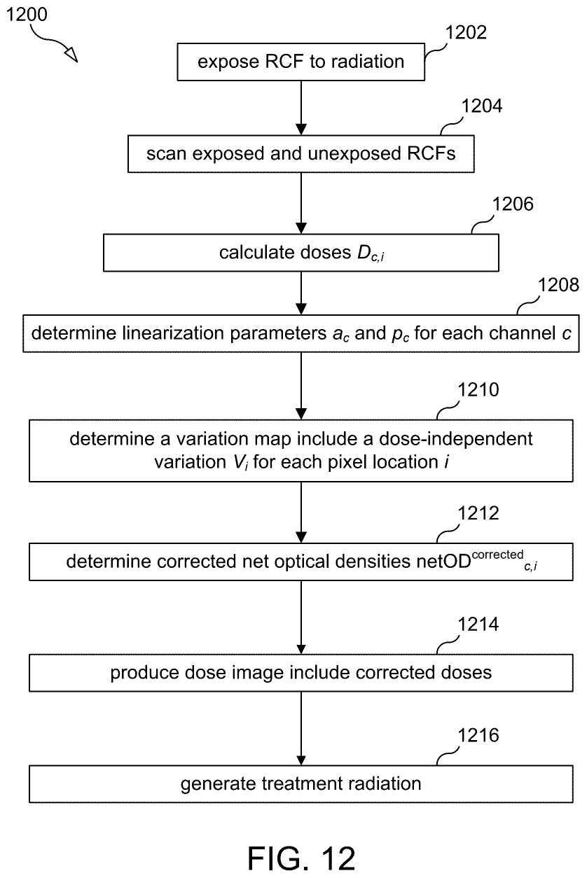

FIG. 12 is a flowchart illustrating an ROL process 1200 according to some aspects. In some aspects, one or more of the steps of the process 1200 may be performed by the system 900. In some aspects, the process 1200 may include a step 1202 of using the LINAC 100 to generate radiation (e.g., radiation beam 103) and expose a radiochromic film (RCF) 902 (e.g., of a film phantom 104) to the radiation. In some aspects, the LINAC 100 and film phantom 104 may be setup as shown in FIG. 1 during the step 1202 of the process 1200. In some aspects, the step 1202 may produce an exposed RCF 902.

In some aspects, as shown in FIG. 12, the process 1200 may include a step 1204 of using a scanner 906 to scan the exposed RCF 902 across multiple color channels to generate a digital image of the exposed RCF 902 with responses of the RCF 902 to a dose of absorbed radiation in the multiple color channels. In some aspects, the multiple color channels may include red, green, and blue channels. In some aspects, the step 1204 may including using the scanner 906 to scan an unexposed RCF 904 across the multiple color channels to generate a digital image of the unexposed RCF 904. In some aspects, the scanner 906 may scan the exposed RCF 902 and the unexposed RCF 904 together. However, this is not required, and, in some alternative aspects, the scanner 906 may scan the exposed RCF 902 and the unexposed RCF 904 separately. In some other alternative aspects, the scanner 906 may scan the exposed RCF 902, and a different scanner may scan the unexposed RCF 904.

In some aspects, as shown in FIG. 12, the process 1200 may include a step 1206 of, for each color channel c of the multiple color channels, for each pixel location i of the digital image of the RCF 902 that was exposed to the radiation generated by the LINAC 100, using a pixel value PV_{c,i} at the pixel location i to calculate a dose D_{c,i} of radiation in the color channel c absorbed by the RCF 902 at a location corresponding to the pixel location i. In some aspects, an optical density of the RCF 902 at the location may have changed in response to the dose of the radiation absorbed by the RCF 902 at the location. In some aspects, for each color channel c of the multiple color channels, for each pixel location i of the digital image of the RCF 902 that was exposed to the radiation, using the pixel value PV_{c,i} at the pixel location i to calculate the dose D_{c,i} of radiation in the color channel c absorbed by the RCF 902 at the location corresponding to the pixel location i in step 1206 may include: (i) using the pixel value PVC_{c,i} at the pixel location i to determine an uncorrected net optical density netOD_{c,i} of the RCF 902 in the color channel c at the location of the RCF 902 corresponding to the pixel location i and (ii) using the uncorrected net optical density netODc,i of the RCF 902 in the color channel c at the location of the RCF 902 corresponding to the pixel location i to calculate the dose D_{c,i} of radiation in the color channel c absorbed by the RCF 902 at the location corresponding to the pixel location i. In some aspects, the uncorrected net optical density netOD_{c,j} may be calculated using the pixel value PV_{c,i} and Equation 5. In some aspects, the dose D_{c,i} may be calculated using netOD_{c,i} and Equation 4. In some aspects, using the pixel value PV_{c,i} at the pixel location i to determine the uncorrected net optical density netOD_{c,i} of the RCF 902 in the color channel c at the location of the RCF 902 corresponding to the pixel location i may include normalizing the pixel value PV_{c,i} at the pixel location i by an averaged pixel value PV_{c,unexp} of a digital image of an unexposed RCF 904 in the color channel c. In some aspects, the unexposed RCF 904 may be from the same batch as the exposed RCF 902, and the averaged pixel value PV_{c,unexp} may relate to a region of interest of the unexposed RCF 904. In some aspects, normalizing the pixel value PV_{c,i} using the averaged pixel value PV_{c,unexp} may be preferred over single-point scaling, such as using the maximum or central dose, as individual points could be affected by local disturbances. However, some alternative aspects may include normalizing the pixel value PV_{c,i} using single-point scaling (e.g., using the maximum or central dose) instead.

In some aspects, as shown in FIG. 12, the process 1200 may include a step 1208 of, for each color channel c of the multiple color channels, determining linearization parameters that minimize a cost function that evaluates an error between expected doses Dᵢ at pixel locations i and the calculated doses D_{c,i} of the radiation in the color channel c absorbed by the RCF 902 at the locations corresponding to the pixel locations i. In some aspects, the linearization parameters for each color channel c may include a scaling value a_{c} and a power value p_{c}. In some aspects, the error evaluated by the cost function may be a mean absolute error between the expected doses Dᵢ at the pixel locations i and the calculated doses D_{c,i} of the radiation in the color channel c absorbed by the RCF 902 at the locations corresponding to the pixel locations i. In some aspects, the cost function may be as shown in Equation 6.

In some aspects, as shown in FIG. 12, the process 1200 may include a step 1210 of generating a variation map including, for each pixel location i of the digital image of the RCF 902, a dose-independent variation value Vᵢ for the pixel location i. In some aspects, generating the variation map may include, for each pixel location i of the digital image of the RCF 902: (i) for each color channel c of the multiple color channels, using the linearization parameters determined for the color channel c to calculate an optimized linearized dose D^{rol}_{c,i} of the radiation in the color channel c absorbed by the RCF 902 at the location corresponding to the pixel location i and (ii) determining the variation value Vᵢ for the pixel location i based on differences between the optimized linearized doses D^{rol}_{c,i} calculated for the multiple color channels c. In some aspects, Equations 10a and 10b may be used to calculate the optimized linearized dose D^{rol}_{c,i}. In some aspects, Equation 9 may be used to determine the variation value Vᵢ.

In some aspects, as shown in FIG. 12, the process 1200 may include a step 1212 of, for each color channel c of the multiple color channels, for each pixel location i of the digital image of the RCF 902, using the pixel value PV_{c,i} for the pixel location i in the color channel c and the variation value Vᵢ for the pixel location i to determine a corrected net optical density netOD^{corrected}_{c,i} of the RCF 902 for the color channel c at the pixel location i. In some aspects, for each color channel c of the multiple color channels, for each pixel location i of the digital image of the RCF 902, using the pixel value PV_{c,i} at the pixel location i in the color channel c and the variation value Vᵢ for the pixel location i to determine a corrected net optical density netOD^{corrected}_{c,i} of the RCF 902 in the color channel c at the location of the RCF 902 corresponding to the pixel location i may include: (i) using the pixel value PV_{c,i} at the pixel location i in the color channel c to calculate a scanned optical density scanODc,i for the pixel location i in the color channel c and (ii) using the scanned optical density scanODc,i for the pixel location i in the color channel c and the variation value Vᵢ for the pixel location i to determine the corrected net optical density netOD^{corrected}_{c,i} of the RCF 902 in the color channel c at the location of the RCF 902 corresponding to the pixel location i.

In some aspects, the corrected net optical density netOD^{corrected}_{c,i} of the RCF 902 in the color channel c at the pixel location i may be determined using the averaged pixel value PV_{c,unexp} of the digital image of the unexposed RCF 904 in the color channel c. In some aspects, determining the corrected net optical density netOD^{corrected}_{c,i} of the RCF 902 for the color channel c at the pixel location i using the averaged pixel value PV_{c,unexp} of the digital image of the unexposed RCF 904 in the color channel c may include: (i) using the averaged pixel value PV_{c,unexp} of the digital image of the unexposed RCF 904 in the color channel c to calculate a scanned optical density scanOD_{c,unexp} of the digital image of the unexposed RCF 904 in the color channel c and (ii) using the scanned optical density scanOD_{c,unexp} of the digital image of the unexposed RCF 904 in the color channel c to determine the corrected net optical density netOD^{corrected}_{c,i} of the RCF 902 in the color channel c at the pixel location i.

In some aspects, the averaged pixel value PV_{c,unexp} and Equation 2 may be used to calculate the scanned optical density scanOD_{c,unexp}. In some aspects, the pixel value PV_{c,i} and Equation 2 may be used to calculate the scanned optical density scanOD_{c,i}. In some aspects, the scanned optical density scanOD_{c,i}, the scanned optical density scanOD_{c,unexp}, the variation value Vᵢ, and Equation 11 may be used to determine the corrected net optical density netOD^{corrected}_{c,i}.

In some aspects, as shown in FIG. 12, the process 1200 may include a step 1214 of producing a dose image including a corrected dose for each color channel c of the multiple color channels at each pixel location i of the digital image of the RCF 902. In some aspects, producing the dose image may include, for each color channel c of the multiple color channels and each pixel location i of the digital image of the RCF 902, using the linearization parameters determined for the color channel c and the corrected net optical density netOD^{corrected}_{c,i} of the RCF 902 for the color channel c at the pixel location i to calculate the corrected dose for the color channel c at the pixel location i. In some aspects, the linearization parameters (e.g., a_{c} and p_{c}), the corrected net optical density netOD^{corrected}_{c,i}, and Equation 12 may be used to determine the corrected dose for the color channel c at the pixel location i. In some aspects, Equation 12 may be based on Equation 4 but uses the linearization parameters a_{c} and p_{c} determined in step 1208 (instead of the scaling factor a and the fixed value ⅔, respectively) and the corrected net optical density netOD^{corrected}_{c,i} determined in step 1212 (instead of the uncorrected net optical density).

In some aspects, as shown in FIG. 12, the process 1200 may include a step 1216 of applying the corrected doses. In some aspects, the application step 1216 may include comparing the corrected doses with the expected doses Dᵢ to determine whether the LINAC 100 meets acceptable performance criteria, and exposing the patient 910 to treatment radiation generated by the LINAC 100 only if the LINAC 100 was determined to meet the acceptable performance criteria. In some aspects, the application step 1216 may include using the corrected doses to recalibrate the LINAC 100 and using the recalibrated LINAC 100 to generate treatment radiation that exposes the patient 910 to a radiation dose distribution in accordance with a patient-specific treatment plan. In some aspects, recalibrating the LINAC 100 may include determining adjustments to the LINAC 100 that would cause the LINAC 100 to generate radiation that would expose the patient 910 to a radiation dose distribution in accordance with an expected dose distribution and making the determined adjustments to the LINAC 100. In some aspects, recalibrating the LINAC 100 may include adjusting the expected dose distribution to match the dose image and using the adjusted expected dose distribution generating the treatment radiation.

In some aspects, step 1103 of the process 1100 and step 1208 of the process 1200 may determine an optimal mathematical function that linearizes and scales a response of the RCF 902 in each of the multiple color channels so that a transformed pixel response of each of the multiple color channels is optimally matched to an expected dose distribution for the RCF 902. In some aspects, step 1105 of the process 1100 and step 1210 of the process 1200 may apply the optimal mathematical function to determine the contribution of the dose-independent portion, wherein removing the dose-independent portion minimizes differences in radiation dose values across the multiple color channels. In some aspects, the dose-independent portions may be in the form of the variation map. In some aspects, steps 1106 and 1107 of the process 1100 and steps 1212 and 1214 of the process 1200 may remove the dose-independent portion from the color channels to produce a dose image that reflects only dose-dependent values.

### Examples

To assess the ability of the ROL method to replicate dose distributions produced by MCD, EBT4 film, which is a type of RCF, was used to measure dose distributions for three treatment plans: open and wedged 10×10 cm fields, and a volumetric modulated arc therapy (VMAT) plan. The comparison was based on dose-difference maps between the traditional MCD method and the ROL method across all three color channels.

An open field was chosen as the simplest example to demonstrate the technique's effectiveness. A wedge field was included to evaluate performance in mid-dose ranges, which are typically obscured within the penumbra. The VMAT plan was selected as a real-world clinical example to assess the method's applicability.

All plans were created in Eclipse (Siemens Healthineers, Erlangen, Germany), and the expected planar doses were exported in Digital Imaging and Communications in Medicine (DICOM) format. During both plan delivery and calibration, the RCF was positioned at a depth of 5 cm within a polystyrene slab, with an additional 5 cm of backscatter material placed behind it. The VMAT test plan included two spherical targets positioned approximately 7 cm apart in the coronal plane at isocenter, with diameters of approximately 4 and 5.5 cm. Each target was prescribed a dose of 300 cGy.

Films were scanned using an Epson® 11000 XL scanner in transparency mode with 16 bits per channel at a resolution of 150 dpi, with all color adjustments disabled. The cross-plane direction of the film was aligned with the scanner's carriage travel direction. A glass plate was placed over the films to ensure they remained flat during scanning.

The one-scan method proposed by Lewis was used to scale the original dose-response curves, and the triple-channel non-uniformity correction method of Micke was subsequently applied. As part of the one-scan method, two reference films were scanned alongside each quality assurance (QA) RCF film: one unexposed and the other exposed to the maximum dose expected in the QA RCF film. Films were physically marked with fiducials based on the room lasers, and these marks were used to register the scanned images. Small registration adjustments were also made in software to optimally align the measured and expected distributions spatially. Ideal spatial coincidence was desired for benchmarking the technique; however, controlled spatial errors were also introduced to assess the method's robustness. Additionally, different threshold values (e.g., z in Equation 6) of 1%, 5%, 10% and 15% were evaluated to assess the effect of threshold selection, and to support the selection of a final threshold value for comparing ROL and MCD dose across all test plans.

To evaluate the robustness of the ROL method against discrepancies between measured and expected dose distributions, the VMAT test case was repeated with two types of errors: (1) a 3 mm spatial positioning error applied in the cross-plane direction, referred to as VMAT-shifted, and (2) a dose delivery error in which the second of the two arcs was prematurely terminated by approximately 25%, referred to as VMAT-partial. This analysis is of particular interest because the expected dose is used in the ROL cost function to optimize the linearization parameters, and any mismatch between the measured film dose and the expected dose can lead to inaccurate or misleading results. Spatial positioning and dose delivery errors are common in clinical QA tests. The ROL technique would therefore be impractical if the resulting dose distribution were strongly affected by such sources of error. These two scenarios were selected as worst-case conditions to evaluate the ROL method's robustness.

To evaluate the ability of the ROL method to detect errors arising from incorrect treatment planning modeling parameters, an analysis was performed using a C-shape test plan based on the American Association of Physicists in Medicine (AAPM) Task Group 119 guidelines. In this case, errors in the multileaf collimator (MLC) modeling, specifically, the dosimetric leaf gap (DLG), were simulated by systematically adjusting the MLC offset in the treatment planning system. This test evaluates the method's sensitivity to beam model discrepancies, such as those introduced during LINAC commissioning or TPS configuration.

The C-shape plan consisted of a PTV surrounding a central cylindrical core. The PTV was an arc-shaped structure with an inner radius of 1.5 cm, an outer radius of 3.7 cm, and a length of 8 cm. The central core, which was separated from the PTV by a 0.5 cm margin, was a 1 cm-radius cylinder with a length of 10 cm. RCF was irradiated with a 6 MV flattened beam using a modular film phantom composed of 2 cm-thick slabs of 15×15 cm Acrylonitrile Butadiene Styrene (ABS) material (density: 1.04 g/cm3), with laser-cut EBT4 film precisely registered within the phantom using a three-pin registration system.

The plan was based on a synthetic computed tomography (CT) dataset, with the origin aligned to the physical center of the film. This ensured accurate origin definition during treatment planning. Irradiations were performed on a Truebeam LINAC (Siemens Healthineers, Erlangen, Germany) with the film positioned in the axial orientation. Setup was performed via CBCT registration and verified using in-room lasers. No additional shifts were introduced—all analysis was based solely on the geometric accuracy of the Cone-Beam Computed Tomography (CBCT) alignment and film registration system.

Dose distributions were calculated using Eclipse v18.0.1.261 (Siemens Healthineers, Erlangen, Germany), with MLC offset values varied from 0.0 to 0.4 mm in 0.1 mm increments. Film registration, including rotation and translation corrections, was executed using custom software based on fiducial markers and phantom pin alignment. Gamma analysis was conducted using a global 2 mm, 3% criterion, with pass rates computed over the region receiving more than 10% of the maximum dose.

While the principal aim was to evaluate the ROL method's sensitivity to small variations in the planning model, context for the results was provided by conducting a parallel evaluation of gamma pass rates between doses predicted by the TPS and those measured via the MCD technique on the same film. The MCD dose was derived using the same procedure described above which included dose response curves, one-scan film patches, and computation of variation maps. Gamma pass rates for each MLC offset were compared between the two methods.

### Examples—Results of Relative Linearization Without Optimization

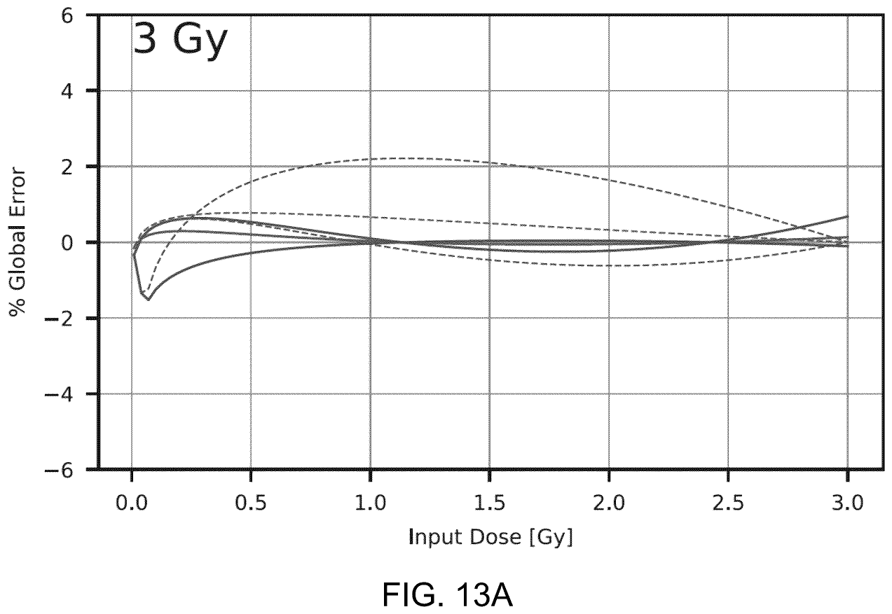

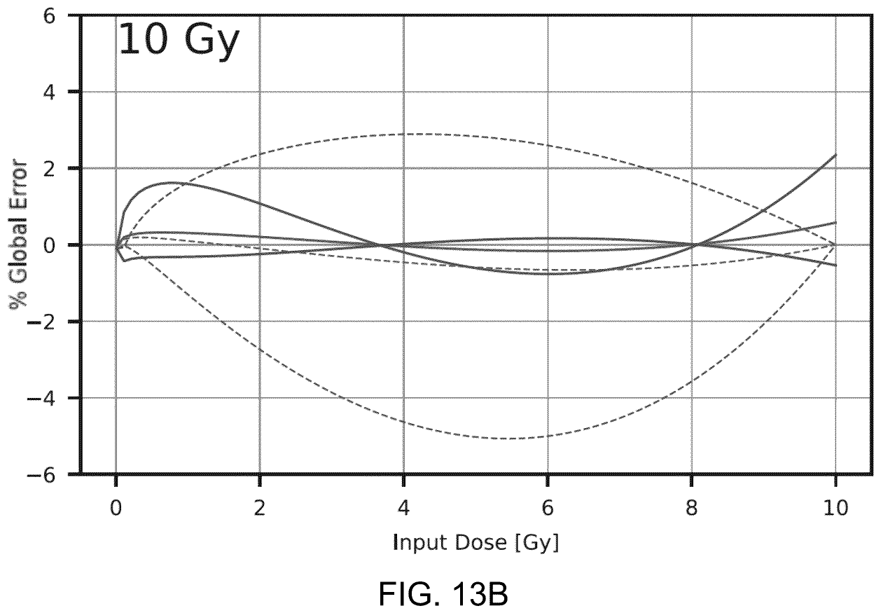

Simulation results using measured EBT4 dose-response curves and the original (non-optimized) linearization method of Devic, applied to dose profiles with maximum doses of 3 and 10 Gy, are shown as dashed lines in FIGS. 13A and 13B, respectively. As shown in FIG. 13A, for the 3 Gy simulation, the blue channel exhibits the largest errors, reaching up to 2% around 1 Gy. In contrast, the red and green channels show relatively low errors, remaining below 1% across nearly the entire dose range. As shown in FIG. 13B, for the 10 Gy simulation, the green channel demonstrates the largest errors, peaking at approximately 5% near 5 Gy. The blue channel shows maximum errors of about 2% around 4 Gy, while the red channel achieves the best linearization, with errors consistently below 1%.

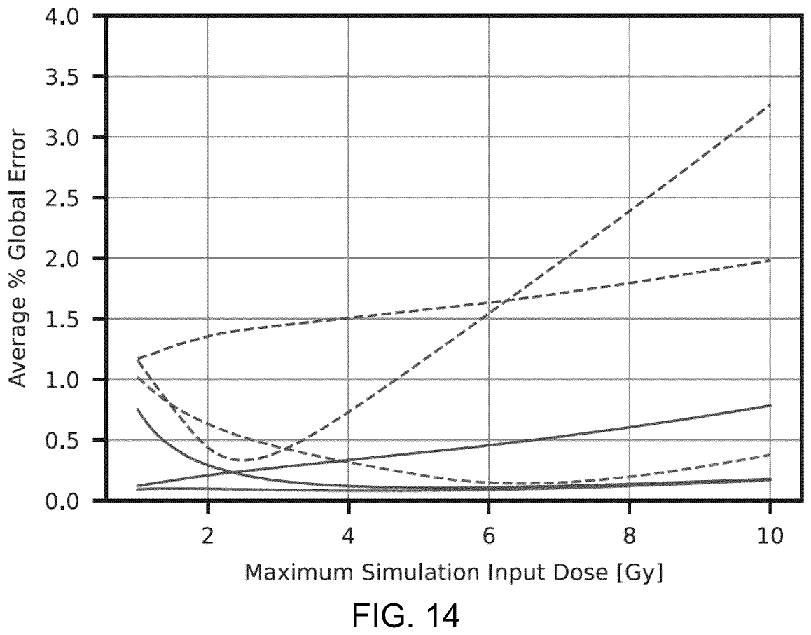

Repeating this type of simulation with datasets featuring different maximum doses ranging from 1 to 10 Gy yielded the average linearization errors shown as the dashed lines in FIG. 14. In FIG. 14, the red channel (dashed red line) exhibits the smallest errors, reaching as low as 0.1% around 7 Gy. The green channel shows its minimum error just below 0.5% near 2 Gy before steadily increasing to 3% at 10 Gy. The blue channel's error gradually increases from approximately 1% at 1 Gy to 2% at 10 Gy.

### Examples—Results of Relative Linearization With Optimization

The optimal power and scale values (a and p in Equation 4) for both 3 and 10 Gy simulations for all three color channels are shown in rows 1 and 2 of Table 1 below. The solid lines in FIGS. 13A and 13B show the results of the ROL method for both the 3 and 10 Gy simulations using these optimized values. Compared to the non-optimized linearization (dashed lines), the ROL approach reduces percentage errors across all dose levels. Similarly, the solid lines in FIG. 14 represent the average global percentage errors observed across multiple optimized linearization simulations with maximum dose values ranging from 1 to 10 Gy. These results demonstrate a substantial reduction in errors for the ROL method across all dose values compared to the non-optimized averages (dashed lines in the same plot).

TABLE 1

| | p | | | a | | |
|---|---|---|---|---|---|---|
| | Red | Green | Blue | Red | Green | Blue |
| Sim, 3 Gy | 0.690 | 0.669 | 0.727 | 1065.5 | 1920.8 | 5940.0 |
| Sim, 10 Gy | 0.657 | 0.531 | 0.753 | 1020.8 | 1498.9 | 6366.9 |
| Open | 0.703 | 0.678 | 0.723 | 1157.0 | 2096.2 | 6614.7 |
| Wedge | 0.675 | 0.639 | 0.691 | 1128.4 | 1970.7 | 6233.6 |
| VMAT | 0.762 | 0.772 | 1.027 | 1204.8 | 2375.9 | 13976.3 |
| VMAT-shifted | 0.754 | 0.769 | 1.025 | 1185.5 | 2352.3 | 13813.8 |
| VMAT-partial | 0.730 | 0.713 | 0.823 | 1307.4 | 2438.0 | 9175.9 |

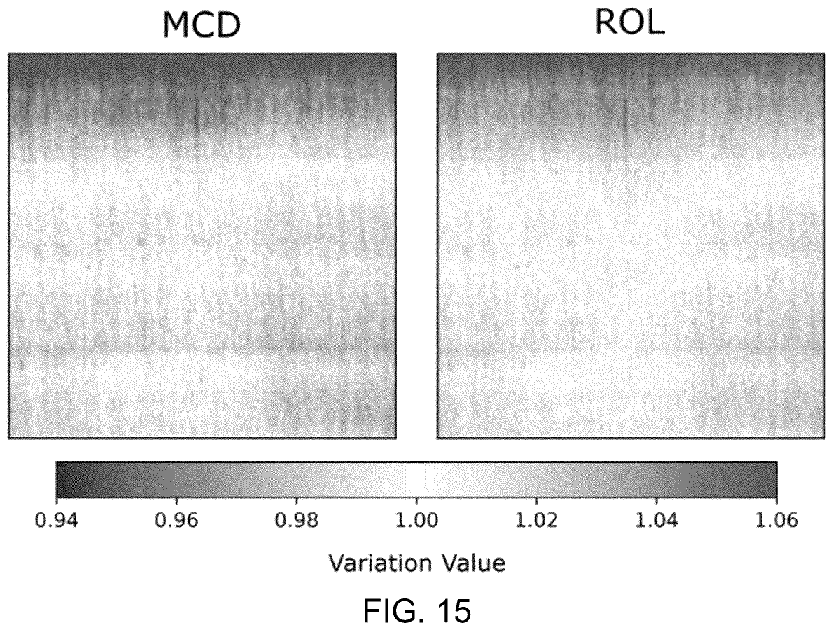

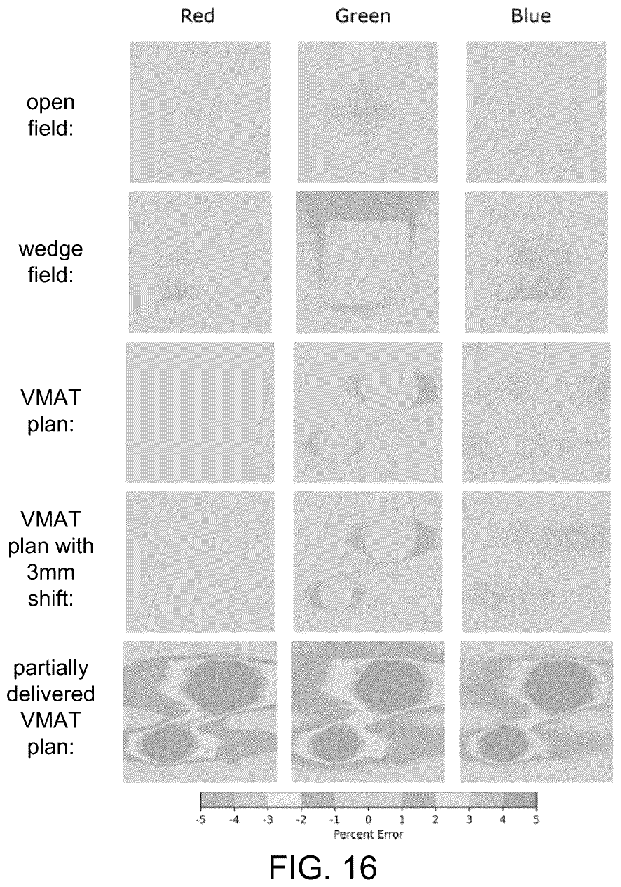

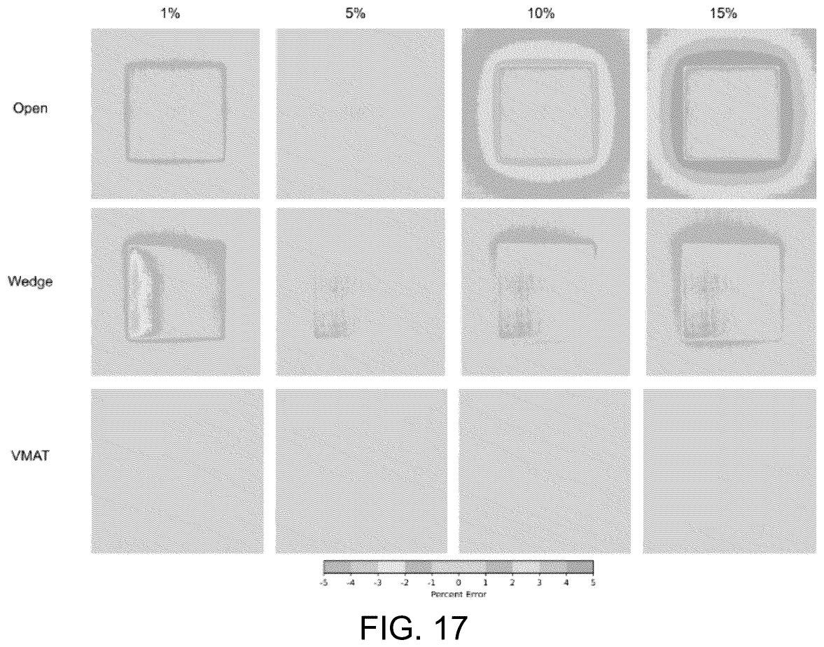

Measured response curves for EBT4 film were used with the MCD method to compute the planar dose across all three color channels for each film, serving as references to evaluate the effectiveness of the new ROL approach. The optimal power (p) and scaling values (a) determined for all test fields are shown in Table 1. The computed variation maps for the wedge field, generated using both MCD and ROL, are presented in FIG. 15. These results demonstrate that ROL can produce variation maps closely matching those obtained with MCD (open and VMAT variation maps were also very similar between the MCD and ROL methods). The percent difference maps comparing the MCD and ROL doses for all test plans are shown in FIG. 16. A threshold of 5% was used for the ROL parameter optimization. This choice was based on the results of the threshold evaluation tests shown in FIG. 17, where 5% produced the best agreement between ROL and MCD.

The average absolute error, and standard deviation of the raw error, evaluated within the low-, middle-, and high-dose regions, are shown in Table 2 below.

TABLE 2

| Test Plan | Channel | Low | Middle | High |
|---|---|---|---|---|
| Open | Red | 0.28 ± 0.09 | 0.15 ± 0.17 | 0.42 ± 0.26 |
| | Green | 0.50 ± 0.22 | 0.37 ± 0.31 | 0.63 ± 0.33 |
| | Blue | 0.23 ± 0.27 | 0.54 ± 0.36 | 0.36 ± 0.43 |
| Wedge | Red | 0.51 ± 0.14 | 0.47 ± 0.35 | 0.70 ± 0.31 |
| | Green | 0.87 ± 0.28 | 0.51 ± 0.37 | 0.37 ± 0.43 |
| | Blue | 0.43 ± 0.26 | 0.71 ± 0.42 | 0.61 ± 0.42 |
| VMAT | Red | 0.16 ± 0.18 | 0.20 ± 0.32 | 0.18 ± 0.22 |
| | Green | 0.31 ± 0.33 | 0.76 ± 0.33 | 0.19 ± 0.24 |
| | Blue | 0.32 ± 0.31 | 0.66 ± 0.37 | 0.38 ± 0.36 |
| VMAT shifted | Red | 0.16 ± 0.16 | 0.13 ± 0.28 | 0.39 ± 0.24 |
| | Green | 0.32 ± 0.33 | 0.79 ± 0.35 | 0.39 ± 0.23 |
| | Blue | 0.28 ± 0.30 | 0.65 ± 0.37 | 0.70 ± 0.36 |
| VMAT partial | Red | 0.92 ± 0.40 | 3.43 ± 1.73 | 10.19 ± 0.59 |
| | Green | 1.20 ± 0.41 | 3.20 ± 1.51 | 10.13 ± 0.71 |
| | Blue | 0.72 ± 0.46 | 2.98 ± 1.65 | 9.81 ± 0.72 |

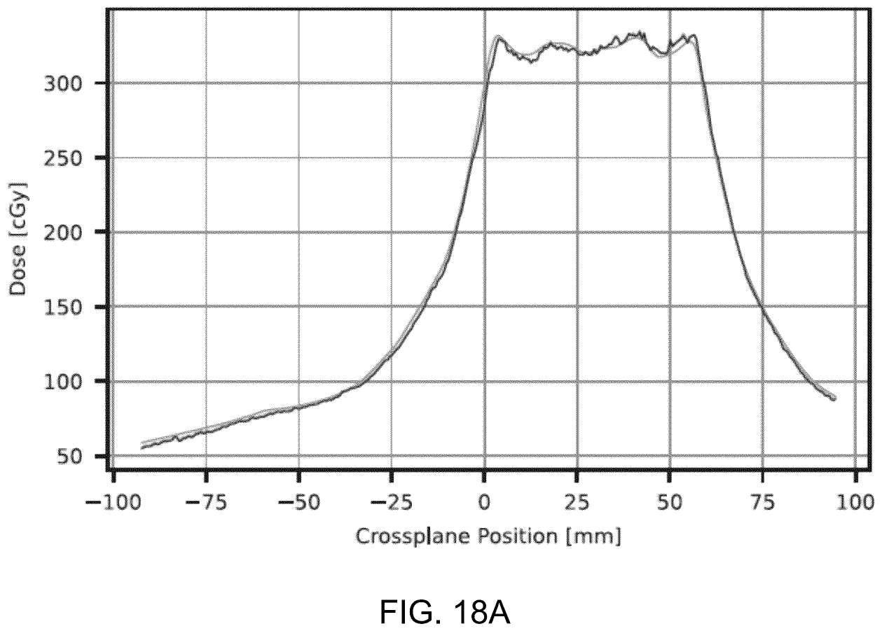

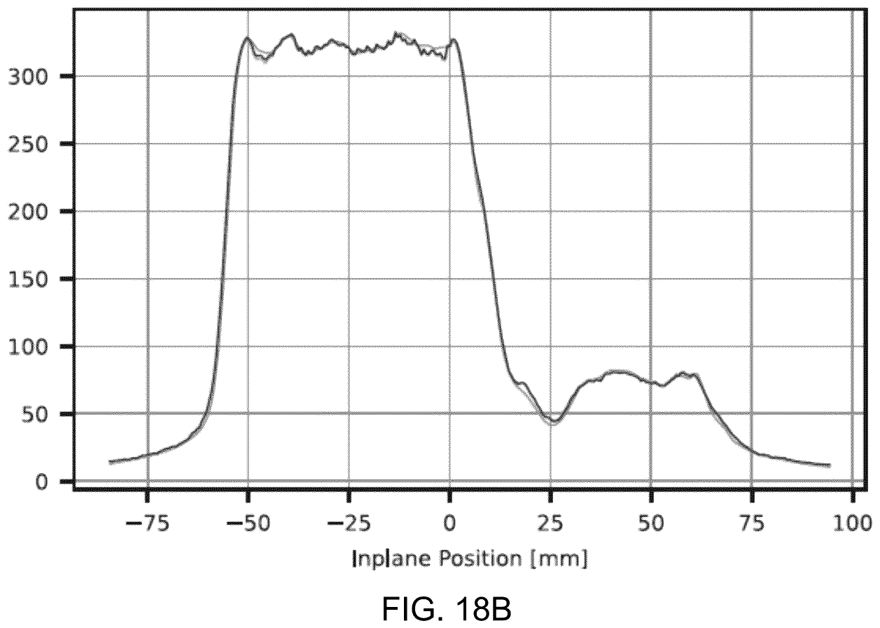

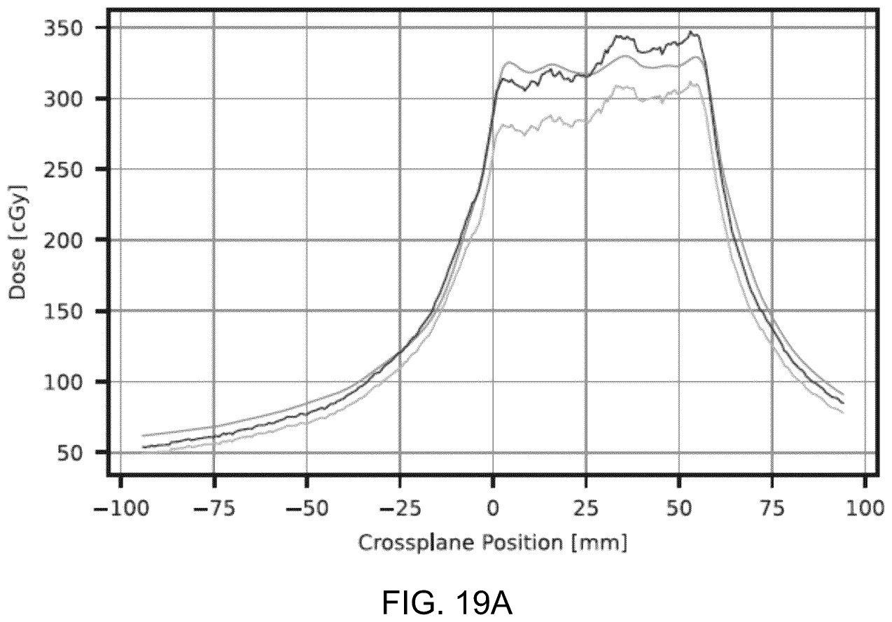

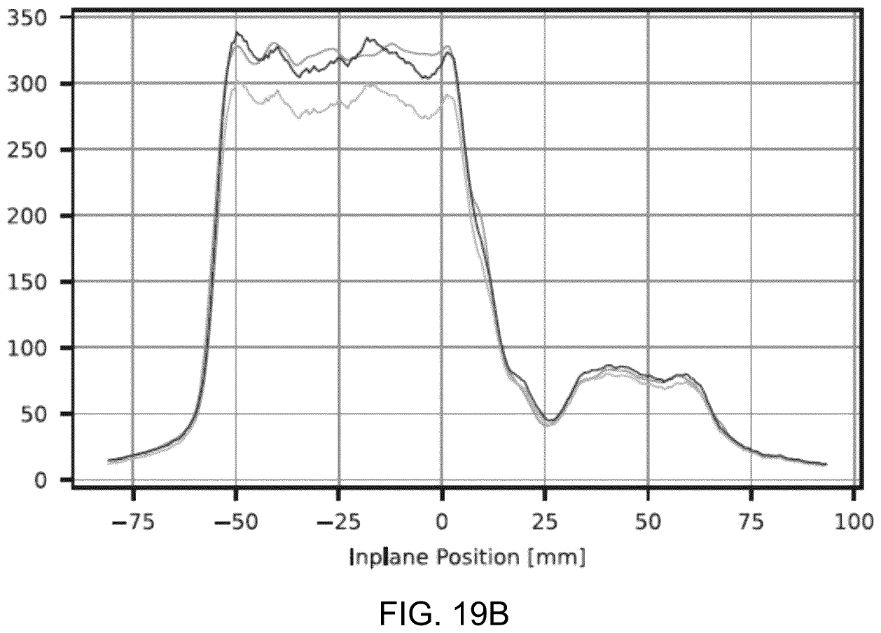

Dose profiles for the VMAT field, computed using both MCD and ROL, are shown in FIGS. 18A and 18B, respectively. These profiles demonstrate excellent agreement between the doses calculated with MCD and the proposed ROL technique. Aside from the partial VMAT, similar levels of agreement were observed in the dose profiles for the open and wedge test plans. Profiles for the partial VMAT plan are shown in FIGS. 19A and 19B. FIGS. 19A and 19B show that the MCD dose is, as expected, considerably lower than the planned dose. The ROL relative dose, which is scaled as part of the method, is closer to the expected dose, but inconsistencies are present between the two due to the fact that the plan was partially delivered and not simply scaled in monitor units delivered.

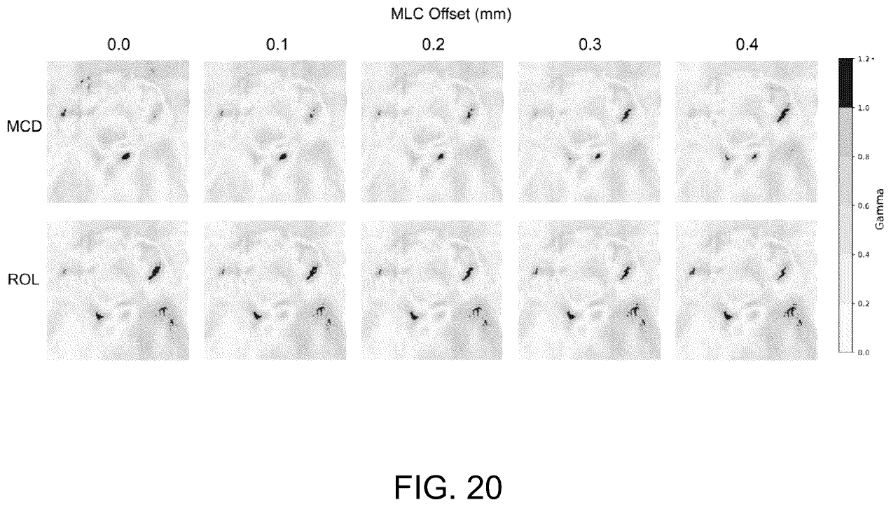

Gamma analysis results from the MLC offset sensitivity tests are shown in FIG. 20 and demonstrate that both MCD and ROL exhibit small but noticeable variations across the different MLC offset values. The corresponding pass rates, summarized in Table 3 below, indicate that the optimal MLC offset was 0.1 mm for MCD and 0.2 mm for ROL. Notably, the value being used clinically was 0.2 mm.

TABLE 3

Gamma pass rate (%)

| MLC Offset (mm) | MCD | ROL |
|---|---|---|
| 0.0 | 99.69 | 99.34 |
| 0.1 | 99.80 | 99.35 |
| 0.2 | 99.79 | 99.39 |
| 0.4 | 99.62 | 99.35 |

The ROL method for radiochromic film dosimetry eliminates the need for dose-response curve measurements. The ROL method uses an improved linearization formula for pixel-to-dose conversion, demonstrating accuracy across the full EBT4 dose range up to 10 Gy. The ROL method incorporates non-uniformity corrections, which achieve high-accuracy film dosimetry.

The results from the simulations performed provide insight into the average linearization errors that can be expected from ROL as a function of the maximum dose delivered to the film. For example, the results shown in FIG. 14 demonstrate that the red channel maintains a low average error of less than 0.25% across all maximum dose levels. The blue channel exhibits higher average errors at lower doses but approaches the red channel's performance around 4 Gy. In contrast, the green channel shows a roughly linear increase in average error with increasing maximum dose, though it remains below 1% even at 10 Gy.

This behavior stands in contrast to the non-optimized linearization, where the accuracy is more sensitive to the maximum dose, and this sensitivity can vary by film type. For instance, Devic reported that linearization for EBT2 and EBT3 films resulted in inaccuracies in the red channel when the maximum dose exceeded 1 Gy. To confirm this, we ran additional simulations using published EBT3 dose-response curves and observed similar trends: the red channel showed the largest errors, with maximum deviations around 4% at 2 Gy, increasing to 7% at 10 Gy. In contrast, the EBT4 non-optimized simulation results presented here in FIG. 14 show excellent red channel linearization (<1% error), with the green channel steadily increasing above 3 Gy and reaching as high as 3% (see dashed green line). By introducing optimization into the linearization process, this sensitivity to maximum dose is effectively eliminated, with all channel errors remaining below 1% regardless of the dose range.

The ROL technique was evaluated by comparing it with established multichannel dosimetry across all color channels for various test cases, including open and wedge fields, as well as a VMAT plan. The results demonstrated that the ROL method successfully solved for the linearization parameters, yielding optimized power values ranging from 0.531 to 1.027. While these values deviate from the fixed exponent of ⅔, they remain within a similar range, suggesting that a variable power offers improved flexibility and accuracy across different dose distributions.

The ROL technique demonstrated the ability to produce variation maps comparable to those generated by MCD (see FIG. 15), as well as corrected dose distributions corrected for non-uniformities (see FIG. 16). This shows that the linearization optimization process, used to independently determine the best linearization parameters for each color channel, yield color channel outputs suitable for accurate variation map computation. The pixel-level agreement between MCD and ROL doses is illustrated in FIGS. 18A and 18B, where the dose profiles from both methods closely match across the field in both the in-plane and cross-plane directions. This strong correlation, observed consistently across all test plans, would not have been possible if the ROL technique were unable to correctly determine variation maps to account for non-uniformities.

As shown in FIG. 16, the red channel demonstrated the best overall agreement between MCD and ROL, with errors generally remaining below 1%. The average error values presented in Table 2 further support this observation: aside from the wedge test, the red channel consistently shows lower average errors and standard deviations across all test plans. The wedge test appearing as an outlier is not unexpected, as it was intentionally selected to evaluate the accuracy of the optimized linearization across a broad range of dose values. In contrast, the open and VMAT plans exhibit a more distinct separation between low- and high-dose regions, which allows the optimization process to more easily match those dominant areas. When dose values are well separated, the algorithm can focus on fitting two primary dose levels. However, when the dose values are more uniformly distributed, such as in the wedge test, it becomes more challenging to find a linearization that accurately fits the entire range. Even in this worst-case scenario, the red channel differences between MCD and ROL remain below 2%, with discrepancies limited to small regions in the toe area of the wedge field.

In some aspects, the linearization optimization cost function (e.g., Equation 6) may include a minimum dose threshold. Without this threshold, the size of the measured film may influence the results, as larger films contained a greater number of out-of-field pixels. The evaluation of different threshold values, shown in FIG. 17, indicated that a 5% threshold was optimal. This threshold effectively excluded pixels just outside the lower transition portion of the penumbra, resulting in more robust and accurate optimization. The threshold selection had little to no noticeable effect on the VMAT plan analysis, which was attributed to the fact that, unlike the open and wedge plans, the VMAT-delivered film had a much lower percentage of pixels below the threshold values. As a result, the optimization was already insensitive to their contribution.

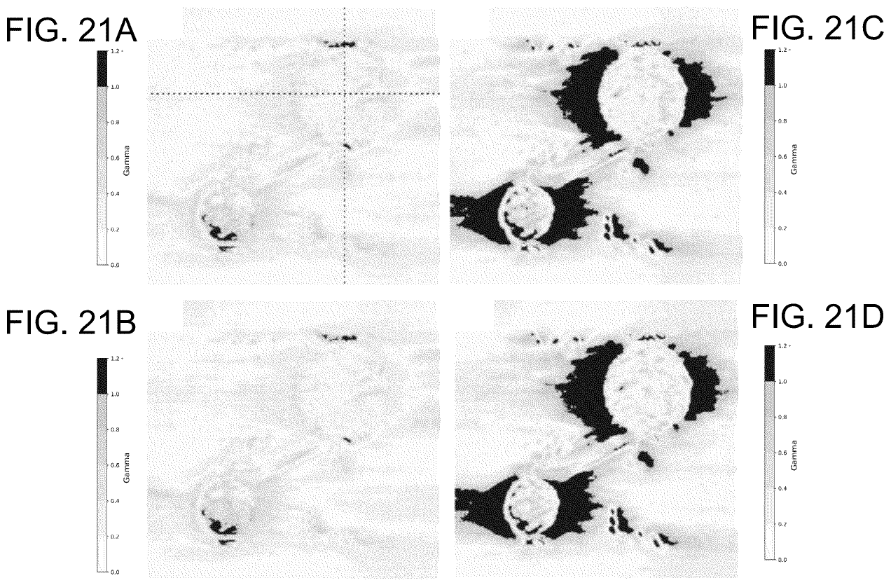

FIGS. 21A-21D illustrate gamma analysis maps (3%, 2 mm) for VMAT expected dose compared against (a) fully delivered film analyzed with MCD, (b) fully delivered film analyzed with ROL, (c) shifted film analyzed with MCD, and (d) shifted film analyzed with ROL, with the crosshairs in FIG. 21A indicating the location of the profiles shown in FIGS. 18A-19B.

The sensitivity tests demonstrated that ROL performs well in the presence of spatial positioning errors between the expected and measured dose distributions. This is evident from the similarity of the gamma maps shown in FIGS. 21C and 21D, which show pass rates of 82.92% and 82.95%, respectively.

Similar trends were observed when small changes were introduced into the treatment planning model. The ROL method was able to resolve differences in DLG values based on variations in MLC offset, indicating that its output reflects sensitivity to the underlying model parameters rather than simply conforming to the expected dose distribution used during optimization. Notably, the MCD results suggested an optimal MLC offset of 0.1 mm, whereas the parallel analysis using ROL methods identified 0.2 mm as optimal, which matched the value previously determined and used clinically by the local physics team in their treatment planning system.

A suitable clinical use case for ROL is routine QA of patient-specific planar dose distributions, where measured film is compared to the expected dose from the TPS, provided that baseline pass rates have been established using MCD during the TPS commissioning and validation process. Another application is the evaluation of subtle changes in dose distribution resulting from modifications to the treatment planning dose model, where ROL can help isolate the dosimetric impact of specific parameter adjustments.

From a clinical implementation standpoint, ROL offers practical advantages, especially in busy clinics with limited resources. Traditional multichannel dosimetry workflows require time-intensive preparation: generating and analyzing dose-response curves for each film batch, performing calibration irradiations, scanning multiple films, and ensuring consistent scanner placement and orientation. Errors introduced by misaligned film placement or inconsistent scanning procedures have been reported, which emphasizes the complexity of current workflows.

The ROL method eliminates the need for batch-specific dose-response measurements and one-scan calibration films. Instead, the ROL method may be carried out using an unexposed RCF, which may be from the same batch and may be scanned and analyzed alongside the QA RCF. The ROL approach simplifies the process, reduces the potential for operator error, and streamlines QA film analysis, making it particularly attractive for high-throughput clinical environments. This, the ROL method is an accurate and efficient approach for converting measured RCF optical density to dose using relative techniques, simplifying the film dosimetry process by eliminating the need for time-consuming calibration curves.

While various embodiments are described herein, it should be understood that they have been presented by way of example only, and not limitation. Thus, the breadth and scope of this disclosure should not be limited by any of the above-described exemplary embodiments. Moreover, any combination of the above-described elements in all possible variations thereof is encompassed by the disclosure unless otherwise indicated herein or otherwise clearly contradicted by context.

Additionally, while the processes described above and illustrated in the drawings are shown as a sequence of steps, this was done solely for the sake of illustration. Accordingly, it is contemplated that some steps may be added, some steps may be omitted, the order of the steps may be re-arranged, and some steps may be performed in parallel. For example, steps 1102 and 1104 of the process 1100 of FIG. 11 may be performed in parallel.

What is claimed is:

1. A method comprising:
for each color channel c of multiple color channels, for each pixel location i of a digital image of a radiochromic film (RCF) that was exposed to radiation generated by a linear accelerator (LINAC), using a pixel value PV_{c,i} at the pixel location i to calculate a dose D_{c,i} of radiation in the color channel c absorbed by the RCF at a location corresponding to the pixel location i, wherein an optical density of the RCF at the location has changed in response to the dose of the radiation absorbed by the RCF at the location;
for each color channel c of the multiple color channels, determining linearization parameters that minimize a cost function that evaluates an error between expected doses Dᵢ at pixel locations i and the calculated doses D_{c,i} of the radiation in the color channel c absorbed by the RCF at the locations corresponding to the pixel locations i;
generating a variation map including, for each pixel location i of the digital image of the RCF, a dose-independent variation value Vᵢ for the pixel location i, wherein generating the variation map comprises, for each pixel location i of the digital image of the RCF:
for each color channel c of the multiple color channels, using the linearization parameters determined for the color channel c to calculate an optimized linearized dose D^{rol}_{c,i} of the radiation in the color channel c absorbed by the RCF at the location corresponding to the pixel location i, and
determining the variation value Vᵢ for the pixel location i based on differences between the optimized linearized doses D^{rol}_{c,i} calculated for the multiple color channels c;
for each color channel c of the multiple color channels, for each pixel location i of the digital image of the RCF, using the pixel value PV_{c,i} for the pixel location i in the color channel c and the variation value Vᵢ for the pixel location i to determine a corrected net optical density netOD^{corrected}_{c,i} of the RCF for the color channel c at the pixel location i; and
producing a dose image comprising a corrected dose for each color channel c of the multiple color channels at each pixel location i of the digital image of the RCF, wherein producing the dose image comprises, for each color channel c of the multiple color channels and each pixel location i of the digital image of the RCF, using the linearization parameters determined for the color channel c and the corrected net optical density netOD^{corrected}_{c,i} of the RCF for the color channel c at the pixel location i to calculate the corrected dose for the color channel c at the pixel location i.

2. The method of claim 1, wherein the multiple color channels include red, green, and blue channels.

3. The method of claim 1, further comprising using the LINAC to generate the radiation and expose the RCF to the radiation.

4. The method of claim 1, further comprising using a scanner to scan the RCF across the multiple color channels to generate the digital image of the RCF with responses of the RCF to the dose of absorbed radiation in the multiple color channels.

5. The method of claim 1, wherein, for each color channel c of the multiple color channels, for each pixel location i of the digital image of the RCF that was exposed to the radiation, using the pixel value PV_{c,i} at the pixel location i to calculate the dose D_{c,i} of radiation in the color channel c absorbed by the RCF at the location corresponding to the pixel location i comprises:
using the pixel value PV_{c,i} at the pixel location i to determine an uncorrected net optical density netOD_{c,i} of the RCF in the color channel c at the location of the RCF corresponding to the pixel location i; and
using the uncorrected net optical density netOD_{c,i} of the RCF in the color channel c at the location of the RCF corresponding to the pixel location i to calculate the dose D_{c,i} of radiation in the color channel c absorbed by the RCF at the location corresponding to the pixel location i.

6. The method of claim 5, wherein using the pixel value PV_{c,i} at the pixel location i to determine the uncorrected net optical density netOD_{c,i} of the RCF in the color channel c at the location of the RCF corresponding to the pixel location i comprises normalizing the pixel value PV_{c,i} at the pixel location i by an averaged pixel value PV_{c,unexp} of a digital image of an unexposed RCF in the color channel c.

7. The method of claim 6, further comprising using a scanner to scan the unexposed RCF across the multiple color channels to generate the digital image of the unexposed RCF.

8. The method of claim 1, wherein the linearization parameters for each color channel c include a scaling value a_{c} and a power value p_{c}.

9. The method of claim 1, wherein the error evaluated by the cost function is a mean absolute error between the expected doses Dᵢ at the pixel locations i and the calculated doses D_{c,i} of the radiation in the color channel c absorbed by the RCF at the locations corresponding to the pixel locations i.

10. The method of claim 1, wherein the corrected net optical density netOD^{corrected}_{c,i} of the RCF in the color channel c at the pixel location i is determined using an averaged pixel value PV_{c,unexp} of a digital image of an unexposed RCF in the color channel c.

11. The method of claim 10, wherein determining the corrected net optical density netOD^{corrected}_{c,i} of the RCF for the color channel c at the pixel location i using the averaged pixel value PV_{c,unexp} of the digital image of the unexposed RCF in the color channel c comprises:
using the averaged pixel value PV_{c,unexp} of the digital image of the unexposed RCF in the color channel c to calculate a scanned optical density scanOD_{c,unexp} of the digital image of the unexposed RCF in the color channel c; and
using the scanned optical density scanOD_{c,unexp} of the digital image of the unexposed RCF in the color channel c to determine the corrected net optical density netOD^{corrected}_{c,i} of the RCF in the color channel c at the pixel location i.

12. The method of claim 1, wherein, for each color channel c of the multiple color channels, for each pixel location i of the digital image of the RCF, using the pixel value PV_{c,i} at the pixel location i in the color channel c and the variation value Vᵢ for the pixel location i to determine a corrected net optical density netOD^{corrected}_{c,i} of the RCF in the color channel c at the location of the RCF corresponding to the pixel location i comprises:
using the pixel value PV_{c,i} at the pixel location i in the color channel c to calculate a scanned optical density scanODc,i for the pixel location i in the color channel c; and
using the scanned optical density scanODc,i for the pixel location i in the color channel c and the variation value Vᵢ for the pixel location i to determine the corrected net optical density netOD^{corrected}_{c,i} of the RCF in the color channel c at the location of the RCF corresponding to the pixel location i.

13. The method of claim 1, further comprising using:
comparing the corrected doses with the expected doses Di and determining that the LINAC meets acceptable performance criteria; and
exposing a patient to treatment radiation generated by the LINAC only if the LINAC was determined to meet the acceptable performance criteria.

14. The method of claim 1, further comprising:
using the dose image to recalibrate the LINAC; and
using the recalibrated LINAC to generate treatment radiation that exposes a patient to a radiation dose distribution in accordance with an expected dose distribution.

15. The method of claim 14, wherein recalibrating the LINAC comprises:
determining adjustments to the LINAC that would cause the LINAC to generate radiation that would expose the patient to a radiation dose distribution in accordance with the expected dose distributions; and
making the determined adjustments to the LINAC.

16. The method of claim 14, wherein recalibrating the LINAC comprises adjusting the expected dose distribution to match the dose image.

17. An apparatus configured to:
for each color channel c of multiple color channels, for each pixel location i of a digital image of a radiochromic film (RCF) that was exposed to radiation generated by a linear accelerator (LINAC), use a pixel value PV_{c,i} at the pixel location i to calculate a dose D_{c,i} of radiation in the color channel c absorbed by the RCF at a location corresponding to the pixel location i, wherein an optical density of the RCF at the location has changed in response to the dose of the radiation absorbed by the RCF at the location;
for each color channel c of the multiple color channels, determine linearization parameters that minimize a cost function that evaluates an error between expected doses Dᵢ at pixel locations i and the calculated doses D_{c,i} of the radiation in the color channel c absorbed by the RCF at the locations corresponding to the pixel locations i;
generate a variation map including, for each pixel location i of the digital image of the RCF, a dose-independent variation value Vᵢ for the pixel location i, wherein generating the variation map comprises, for each pixel location i of the digital image of the RCF:
for each color channel c of the multiple color channels, using the linearization parameters determined for the color channel c to calculate an optimized linearized dose D^{rol}_{c,i}, of the radiation in the color channel c absorbed by the RCF at the location corresponding to the pixel location i, and
determining the variation value Vᵢ for the pixel location i based on differences between the optimized linearized doses D^{rol}_{c,i} calculated for the multiple color channels c;
for each color channel c of the multiple color channels, for each pixel location i of the digital image of the RCF, use the pixel value PV_{c,i} for the pixel location i in the color channel c and the variation value Vᵢ for the pixel location i to determine a corrected net optical density netOD^{corrected}_{c,i} of the RCF for the color channel c at the pixel location i; and
produce a dose image comprising a corrected dose for each color channel c of the multiple color channels at each pixel location i of the digital image of the RCF, wherein producing the dose image comprises, for each color channel c of the multiple color channels and each pixel location i of the digital image of the RCF, using the linearization parameters determined for the color channel c and the corrected net optical density netOD^{corrected}_{c,i} of the RCF for the color channel c at the pixel location i to calculate the corrected dose for the color channel c at the pixel location i.

18. The apparatus of claim 17, wherein the multiple color channels include red, green, and blue channels.

19. The apparatus of claim 17, wherein the apparatus is further configured to use the LINAC to generate the radiation and expose the RCF to the radiation.

20. The apparatus of claim 17, wherein, for each color channel c of the multiple color channels, for each pixel location i of the digital image of the RCF that was exposed to the radiation, using the pixel value PV_{c,i} at the pixel location i to calculate the dose D_{c,i} of radiation in the color channel c absorbed by the RCF at the location corresponding to the pixel location i comprises:
using the pixel value PV_{c,i} at the pixel location i to determine an uncorrected net optical density netOD_{c,i} of the RCF in the color channel c at the location of the RCF corresponding to the pixel location i; and
using the uncorrected net optical density netOD_{c,i} of the RCF in the color channel c at the location of the RCF corresponding to the pixel location i to calculate the dose D_{c,i} of radiation in the color channel c absorbed by the RCF at the location corresponding to the pixel location i.

21. The apparatus of claim 20, wherein using the pixel value PV_{c,i} at the pixel location i to determine the uncorrected net optical density netOD_{c,i} of the RCF in the color channel c at the location of the RCF corresponding to the pixel location i comprises normalizing the pixel value PV_{c,i} at the pixel location i by an averaged pixel value PV_{c,unexp} of a digital image of an unexposed RCF in the color channel c.

22. The apparatus of claim 17, wherein the linearization parameters for each color channel c include a scaling value a_{c} and a power value p_{c}.

23. The apparatus of claim 17, wherein the error evaluated by the cost function is a mean absolute error between the expected doses Dᵢ at the pixel locations i and the calculated doses D_{c,i} of the radiation in the color channel c absorbed by the RCF at the locations corresponding to the pixel locations i.

24. The apparatus of claim 17, wherein the corrected net optical density netOD^{corrected}_{c,i} of the RCF in the color channel c at the pixel location i is determined using an averaged pixel value PV_{c,unexp} of a digital image of an unexposed RCF in the color channel c.

25. The apparatus of claim 24, wherein determining the corrected net optical density netOD^{corrected}_{c,i} of the RCF for the color channel c at the pixel location i using the averaged pixel value PV_{c,unexp} of the digital image of the unexposed RCF in the color channel c comprises:
using the averaged pixel value PV_{c,unexp} of the digital image of the unexposed RCF in the color channel c to calculate a scanned optical density scanOD_{c,unexp} of the digital image of the unexposed RCF in the color channel c; and
using the scanned optical density scanOD_{c,unexp} of the digital image of the unexposed RCF in the color channel c to determine the corrected net optical density netOD^{corrected}_{c,i} of the RCF in the color channel c at the pixel location i.

26. The apparatus of claim 17, wherein, for each color channel c of the multiple color channels, for each pixel location i of the digital image of the RCF, using the pixel value PV_{c,i} at the pixel location i in the color channel c and the variation value Vᵢ for the pixel location i to determine a corrected net optical density netOD^{corrected}_{c,i} of the RCF in the color channel c at the location of the RCF corresponding to the pixel location i comprises:
using the pixel value PV_{c,i} at the pixel location i in the color channel c to calculate a scanned optical density scanODc,i for the pixel location i in the color channel c; and
using the scanned optical density scanODc,i for the pixel location i in the color channel c and the variation value Vᵢ for the pixel location i to determine the corrected net optical density netOD^{corrected}_{c,i} of the RCF in the color channel c at the location of the RCF corresponding to the pixel location i.

27. The apparatus of claim 17, wherein the apparatus is further configured to compare the corrected doses with the expected doses Di and determine whether the LINAC meets acceptable performance criteria, wherein a patient is exposed to treatment radiation generated by the LINAC only if the LINAC was determined to meet the acceptable performance criteria.

28. A system comprising:
the apparatus of claim 17;
the LINAC; and
the scanner.

29. A method comprising:
scanning a radiochromic film (RCF) across multiple color channels to generate a digital image, wherein an optical density of the RCF changes as a result of radiation exposure, the RCF contains an area exposed to a pattern of radiation doses, and the digital image includes responses of the RCF in multiple color channels;
determining an optimal mathematical function that linearizes and scales a response of the RCF in each of the multiple color channels so that a transformed pixel response of each of the multiple color channels is optimally matched to an expected dose distribution for the RCF;
applying the optimal mathematical function to determine a contribution of the dose-independent portion, wherein removing the dose-independent portion minimizes differences in radiation dose values across the multiple color channels; and
removing the dose-independent portion from the color channels to produce a dose image that reflects only dose-dependent values.
